(19) United States
(12) Patent Application Publication
(10) Pub. No.: US 2015/0125901 A1
(43) Pub. Date: May 7, 2015

(54) BIOCONVERSION PROCESSES AND APPARATUS

(71) Applicant: Microvi Blotech, Inc., Hayward, CA (US)

(72) Inventors: Fatemeh Razavi-Shirazi, Hayward, CA (US); Mohammad Ali Dorri, Milpitas, CA (US); Ameen(nmn) Razavi, Fremont, CA (US)

(21) Appl. No.: 14/407,799

(22) PCT Filed: Jun. 14, 2013
(86) PCT No.: PCT/US13/46018

§ 371 Date: Dec. 12, 2014

Related U.S. Application Data
(60) Provisional application No. 61/689,924, filed on Jun. 15, 2012.
(60) Provisional application No. 61/689,943, filed on Jun. 15, 2012.

(51) Int. Cl. C02F 3/34; C12M 1/12; C12P 39/00

(52) U.S. Cl. CPC C02F 3/348; C12P 39/00; C12M 25/14; C02F 2003/001
USPC 435/42; 435/289.1; 210/616

(57) ABSTRACT

Bioconversion processes are disclosed in which two or more biocatalysts including microorganisms or isolated enzymes that are substantially irreversibly retained in the interior of an open, porous, highly hydrophilic polymer are used in a common aqueous medium. In one exemplary embodiment, one biocatalyst produces a chemical product that is a substrate to at least one other biocatalyst. In another exemplary embodiment, the feed includes two or more substrates and one biocatalyst bioconverts at least one substrate and another biocatalyst bioconverts at least one other substrate. This aspect is particularly useful for treating water including disparate contaminants by metabolic degradation in a bioreaction zone including multiple types of biocatalysts.

### CROSS-REFERENCE TO RELATED APPLICATIONS

Priority is claimed to U.S. Provisional Patent Applications Nos.:
61/689,924, filed on Jun. 15, 2012, and
61/689,943, filed on Jun. 15, 2012,
   each of which is hereby incorporated by reference in its entirety. A right is hereby reserved to have patentability determinations made on the basis of the applicable sections of Public Law 112-29.

### FIELD OF THE INVENTION

This invention pertains to processes and apparatus for bioconversion of at least one substrate to produce at least one chemical product using two or more biocatalysts in a common aqueous medium which biocatalysts comprise microorganisms or isolated enzymes (bioactive materials) that are substantially irreversibly retained in the interior of an open, porous, highly hydrophilic polymer.

### BACKGROUND

Metabolic processes have long been proposed for anabolic and catabolic bioconversions. Microorganisms of various types have been proposed for these bioconversions and include bacteria and archaea, both of which are prokaryotes; fungi; and algae. Metabolic processes are used by nature, and some have been adapted to use by man for millennia for anabolic and catabolic bioconversions ranging from culturing yogurt and fermentation of sugars to produce alcohol to treatment of water to remove contaminants. Metabolic processes offer the potential for low energy consumption, high efficiency bioconversions in relatively inexpensive processing equipment and thus may be and are often viable alternatives to chemical synthesis and degradation methods. Often anabolic processes can use raw materials that are preferred from a renewable or environmental standpoint but are not desirable for chemical synthesis, e.g., the conversion of carbon dioxide to biofuels and other bioproducts. Catabolic bioconversions can degrade substrates and have long been used for wastewater treatment. Considerable interests exist in improving metabolic processes for industrial use and expanding the variety of metabolic process alternatives to chemical syntheses and degradations.

Various challenges affecting the adoption of a metabolic process exist and are varied depending upon the nature of the feed and the intended objective of the metabolic process. By way of illustration of the scope over which the challenges can exist several scenarios are presented. One scenario pertains to the treatment of water from various sources such as ground water, surface water, municipal waste water and industrial waste water, can contain contaminants that adversely affect its quality. Removal of these contaminants may be desired such that the water can be used for a desired purpose, e.g., for drinking or industrial use or discharged to the environment or for remediation. In many instances, the water contains a number of disparate contaminants.

Numerous processes have been proposed for removal of certain contaminants such as reverse osmosis, ion exchange, chemical treatment and biological treatment. Where the water contains disparate contaminants, treatment can become complex and expensive due to the number of different unit operations required to achieve sought reduction of each of the contaminants. The water to be treated may, for instance, contain metals, semi-metals, nitrates, perchlorates, organics, ammonia, biocides, and the like. Not only might different unit operations be required for removal of each type of contaminant, but also one contaminant may adversely affect a unit operation to remove another contaminant.

The complexity of the treatment processes can render such processes not viable for use where small volumes of water are being treated, e.g., wells providing water for individual sites such as a home or business. Similarly, temporary treatment facilities such as at a remediation site or at oil and natural gas production operations may not be needed for a time sufficient to amortize the cost of the facility. Thus, any such facility should preferably be capable of being moved from site to site. However, the number of unit operations required to treat a water source can pose difficulties in providing a mobile facility at a reasonable expense.

An additional problem with treatment facilities intended for individual site is that maintenance of the treatment facilities needs to be minimal and not complex to reduce operating costs, to avoid the need for highly trained individuals and to avoid downtime. Processes such as ion exchange and chemical treatment require scheduled operations. And biological treatment almost always requires periodically removing cell debris and other solids.

A particularly challenging source of water to treat is “produced water” generated from any oil and natural gas production operations. Typically the produced water is generated in large quantities, and at the early stages of development of a well, 50,000 or more gallons of produced water may be generated. Produced water is contaminated with significant concentrations of contaminants including hydrocarbons and inorganic salts that naturally occur in the strata from which the hydrocarbons are obtained. Produced water may also include man-made contaminants that are introduced into the well hole such as drilling mud, friction reduction chemicals, artificial lubricants, polymer breaking agents, biocides, viscosifying agents, cross-linking catalysts, anticorrosion agents, anti-icing agents and “frac flow back water” which contains spent fracturing fluids. These contaminants must be removed prior to water reuse or discharge to the environment.

Moreover, with the plethora of organic contaminants, the produced water can be attractive for contamination by microorganisms. The microorganisms, if the water is introduced into the subterranean reservoirs can result in undesired biological activity and can result in generation of acids that attack equipment and piping. For instance, U.S. Published Patent Application No. 20090127210 discloses treating frac water with biocide to kill and prevent the growth of microorganisms that degrade additives, namely viscosifiers, and cause problems in the producing reservoir. U.S. Published Patent Application No. 20110056693 discloses the use of rotenone to prevent or inhibit the deleterious effects of sulfate reducing bacteria in aqueous streams. Sulfate reducing bacteria produce hydrogen sulfide which can attack metals and thus reduce service life of piping and equipment. See also, U.S. Published Patent Application No. 20100307757 also addressing the sulfate reducing bacteria problem using sodium hypochlorite and additives.

Other illustrative types of challenges pertain to anabolic processes, for instance, to bioconvert substrates into biochemicals and biofuels. One such challenge is to determine biocatalysts that have the selectivity and productivity to the sought chemical product to provide an economically feasible, commercial-scale plant. Another challenge is for the biocatalyst to achieve a high conversion of substrate in a feedstock to the sought chemical product. Yet further challenges include enabling the biocatalyst to tolerate other components in the feedstocks to avoid undue capital and operating expense to purify the feedstocks, to tolerate the products of the bioconversion itself, and to avoid deactivation of the biocatalyst during the duration of the bioconversion process. Moreover, in some instances to provide a bioconversion process that will produce a product that is economically competitive with a product synthesized from fossil hydrocarbons, the bioconversion needs to be conducted in a continuous mode including the separation of product from the biocatalyst.

One suggested approach to solving challenges within this wide scope affecting anabolic and catabolic bioprocesses has been to genetically engineer the microorganism to increase selectivity and productivity or to increase tolerance to other components in the feed or to products from the bioconversion.

Another potential approach has been to use more than one type of microorganism. Mixed culture broths can pose practical difficulties as microorganisms can compete for the same substrate and can affect each other's growth and are difficult to control. Accordingly, the use of two or more sequential bioreactors has been proposed where in one bioreactor a microorganism is used in an aqueous medium containing at least one substrate to produce at least one chemical product, and after removal of the first microorganism, in a second, subsequent bioreactor, another microorganism is contacted with the aqueous medium from the preceding bioreactor to produce at least one second chemical product from either a chemical product from the first bioreactor or at least one unconsumed substrate from the preceding bioreactor. The removal of microorganisms between stages is essential where the microorganisms compete for nutrients, present competition concerns and the carried over microorganism is more robust and increases its population more rapidly than that of the microorganism desired in the subsequent bioreactor.

The use of sequential bioreactors enables the duration of the metabolic process in each bioreactor to be controlled to achieve a desired overall conversion of substrate to chemical product. Thus, such proposed processes entail the need for plural bioreactors and associated instrumentation and support equipment and the need for effective separation of the microorganism from the aqueous medium prior to being introduced into the subsequent bioreactor. Additionally, a portion of the aqueous medium is lost with the separated microorganism. Although the amount of loss may be minor, the magnitude of the loss can be appreciable where large volumes of production occur due to the loss of substrate and other nutrients. Further, the lost aqueous medium adds to the waste treatment load.

Eiteman, et al., in United States patent application publication No. 2010/0129883 A1 have proposed methods for producing a biochemical by concurrently contacting the substrate, a hydrolyzed lignocellulosic, with a plurality of sugar selective cells under conditions to allow the plurality of sugar-selective cells to produce the biochemical. More specifically they disclose genetic modification, or engineering, of the microorganism such that one microorganism can metabolize one sugar, but not the other, and then a second microorganism that can metabolize the second sugar, but not the first sugar. Even so, they propose that sequential reactors be used with the first to reduce the acetate content of the hydrolysate prior to being passed to a second stage.

Contag in United States patent application publication no. 2012/0124898 A1 discloses co-culturing photosynthetic polysaccharide-producing microorganisms with polysaccharide-consuming, biofuel producing, non-photosynthetic microorganisms to produce a bio fuel.

### SUMMARY

In accordance with the processes of this invention, two or more biocatalysts can be used in the same aqueous medium without undue competition among microorganisms used in the biocatalysts to effect different metabolic conversions. The biocatalysts comprise microorganisms that are substantially irreversibly retained in the interior of an open, porous, highly hydrophilic polymer, and an essential absence of debris generation from metabolic activity of the microorganisms during the metabolic bioconversion. Hence, process flexibility is provided to address challenges in both anabolic and catabolic processes.

In its broad aspects, the processes of this invention comprise:
a. introducing at least one substrate into a bioreactor containing an aqueous medium wherein the aqueous medium contains at least two biocatalysts wherein:
   i. at least one of the biocatalysts is capable of bioconverting at least one substrate to at least one of an intermediate chemical and a sought chemical product,
   ii. at least one other biocatalyst is capable of bioconverting at least one substrate or intermediate chemical to a chemical product or intermediate chemical product,
   iii. at least one of said biocatalysts having
      a solid structure of hydrated hydrophilic polymer defining an interior structure having a plurality of interconnected major cavities having a smallest dimension of between about 5 and 100 microns and an HEV of at least about 1000, preferably at least about 5000, and
      a population of microorganisms substantially irreversibly retained in the interior structure, said microorganisms being in a concentration of at least about 60 grams per liter based upon the volume defined by the exterior of the solid structure when fully hydrated, and
   iv. at least one of the biocatalysts provides a chemical product;
b. maintaining the aqueous medium under metabolic conditions suitable for the bioconversion of said at least one substrate to at least one chemical product, and
c. recovering said at least one chemical product from the aqueous medium.

Preferably each of the biocatalysts comprise microorganisms substantially irreversibly retained therein. Preferably the biocatalysts have an external structure that does not permit exogenous microorganisms to enter the interior structure of the biocatalyst. The processes of this invention may be batch, semi-batch or continuous.

The at least one substrate may be provided initially or may be provided intermittently or continuously during the bioconversion. The substrate may be one or more of gaseous and liquid substrate. In the processes of this invention wherein a biocatalyst provides an intermediate chemical, such intermediate chemical may be suitable as substrate for at least one other bioactive material. In one embodiment, two or more microorganisms are irreversibly retained in the same porous matrix but at different locations.

In one preferred embodiment, the processes of this invention are used for treating water containing disparate contaminants, which processes comprise:
(i) continuously introducing said water into a bioreaction zone containing a plurality of biocatalysts;
(ii) contacting the water with said biocatalysts under metabolic conditions for a time sufficient to reduce the concentration of the disparate contaminants; and
(iii) withdrawing water having a reduced concentration of disparate contaminants from the bioreaction zone containing a plurality of disparate contaminants,
   wherein in said bioreaction zone a portion of the biocatalysts have substantially irreversibly retained therein one type of microorganism adapted to metabolically degrade at least one disparate contaminant, and at least one other portion of the biocatalysts have substantially irreversibly retained therein another type of microorganism adapted to metabolically degrade at least one other disparate contaminant, and wherein said biocatalysts comprise
a solid structure of hydrated hydrophilic polymer defining an interior structure having a plurality of interconnected major cavities having a smallest dimension of between about 5 and 100 microns and an HEV of at least about 1000, preferably at least about 5000, and
a population of microorganisms substantially irreversibly retained in the interior structure, said microorganisms being in a concentration of at least about 60 grams per liter based upon the volume defined by the exterior of the solid structure when fully hydrated.

The preferred processes of this aspect of the invention treat water in which the disparate contaminants comprise at least one of metalates, nitrates and perchlorates that are subjected to reductive metabolic degradation and at least one of hydrocarbon and alkanol of from about 1 to 6 carbon atoms that are subjected to oxidative metabolic degradation, and other contaminants may be present. The metalates frequently comprise one or more oxyanions, hydroxyls or salts of boron, arsenic, selenium, radium, uranium, tungsten, molybdenum, chromium, and manganese. The water contacted with the biocatalysts preferably contains at least about 1, and often at least about 2, say, 4 to 10 or more, milligrams of free oxygen per liter to provide for oxidative metabolic degradation. The biocatalysts often enable the reductive metabolic degradation in the presence of some free oxygen in the water. In one aspect of the invention, the water to be treated is produced water. In another aspect of the invention, the water to be treated is ground water that has been contaminated by subterranean fracturing for fossil fuel production, e.g., oil or gas wells or coal mining. In one preferred embodiment, the bioreaction zone is a mobile bioreactor. In another preferred embodiment, the bioreaction zone is a point of use assembly, and most preferably a point of use assembly for home or business or single building use.

In another preferred embodiment, portions of different biocatalysts are intermixed in the bioreaction zone. In some instances, the biocatalysts are in an expanded or fluidized bed. Where the water contains microorganisms, preferably the biocatalysts are intermittently washed to remove at least a portion of the contaminating microorganisms from the surfaces of the biocatalysts. Where the water contains metalates, the metals are reduced and form precipitates that are often retained in the biocatalysts. The use of a subsequent ultrafiltration unit operation can be used to assure removal of any of these precipitates that pass into the treated water. Where the precipitates are retained in the biocatalysts, density may be used to remove loaded biocatalysts containing metal precipitates from the bioreaction zone. Fresh biocatalysts capable of metabolic reduction of metalates can be added to the bioreaction zone to maintain a desired activity in the reaction zone. Nutrients for the microorganisms may be contained in the water to be treated, added to the bioreactor or may be incorporated into the biocatalysts for access by the microorganisms when required.

The apparatus of this invention broadly pertain to bioreactors for the bioconversion of at least one substrate to at least one chemical product comprising:
a. a vessel defining an interior volume;
b. an aqueous medium contained in at least a portion of the interior volume of the vessel; and
c. at least two biocatalysts distributed within the aqueous medium at least one of which comprises:
   a solid structure of hydrated hydrophilic polymer defining an interior structure having a plurality of interconnected major cavities having a smallest dimension of between about 5 and 100 microns and an HEV of at least about 1000, preferably at least about 5000, and
   a population of microorganisms substantially irreversibly retained in the interior structure, said microorganisms being in a concentration of at least about 60 grams per liter based upon the volume defined by the exterior of the solid structure when fully hydrated.

Preferably each of the biocatalysts contains microorganisms irreversibly retained therein. In this preferred embodiment, the porous matrices may be intermixed or physically separated. The biocatalysts may form a structure (e.g., fixed or packed) in the vessel or may be freely dispersed within the aqueous medium. Alternatively, the biocatalyst may be a layered biocatalyst containing at least two different regions, each with microorganisms irreversibly retained therein. In some instances the layered biocatalyst may contain a type of microorganism in one region that controls the composition and quantity of a bioproduct to another region or may change the environment in another region, e.g. by changing pH, redox potential or the like of the aqueous medium in the environment proximate to the other region. Where the biocatalysts are freely dispersed, the interior volume of the vessel may contain fluid permeable barriers that are substantially impermeable to the biocatalysts to segment biocatalysts containing different microorganisms.

### BRIEF DESCRIPTION OF THE DRAWINGS

FIG. 1 is a schematic representation of an apparatus in accordance with this invention containing different, freely dispersed biocatalysts.

FIG. 2 is a schematic representation of an apparatus in accordance with this invention containing a stationary biocatalyst which also serves as a baffle to separate different freely-dispersed biocatalyst.

### DETAILED DESCRIPTION

All patents, published patent applications and articles referenced in this detailed description are hereby incorporated by reference in their entireties.

### Definitions

As used herein, the following terms have the meanings set forth below unless otherwise stated or clear from the context of their use.

The use of the terms “a” and “an” is intended to include one or more of the element described. Lists of exemplary elements are intended to include combinations of one or more of the element described. The term “may” as used herein means that the use of the element is optional and is not intended to provide any implication regarding operability.

Adhering to the solid structure of the biocatalyst means that the bioactive material is located in cavities in the interior of the biocatalyst and is substantially irreversibly retained therein although extraordinary conditions and treatments (i.e., not normal bioconversion conditions for bioconversion using the bioactive material) might be able in some instances to cause the bioactive material to exit the biocatalyst. Adhering includes surface attachment to the polymer forming the walls of the porous matrix as well as where the bioactive material are retained microorganisms that are proximate to a polymeric surface, e.g., within about 10 or 20 microns, but not directly contacting the surface. Adhering thus includes physical and electrostatic adherence. In some instances, the polymer used to make the biocatalyst may become embedded in the extracellular polymeric substance around a cell or even in or on the cell wall of the microorganism.

Bioactive material is one or both of microorganisms and isolated enzymes.

Bioconversion activity is the rate of consumption of substrate per hour per gram of bioactive material. Where an increase or decrease in bioconversion activity is referenced herein, such increase or decrease is ascertained under similar bioconversion conditions including concentration of substrate and product in the aqueous medium. Bioconversion activity to bioproduct is the rate of production of the bioproduct per hour per gram of bioactive material.

Biofilm means an aggregate of microorganisms embedded within an extracellular polymeric substance (EPS) generally composed of polysaccharides, and may contain other components such as one or more of proteins, extracellular DNA and the polymer used to make the biocatalyst. The thickness of a biofilm is determined by the size of the aggregate contained within a continuous EPS structure, but a continuous EPS structure does not include fibrils that may extend between separated biofilms. In some instances, the biofilm extends in a random, three dimensional manner, and the thickness is determined as the maximum, straight line distance between the distal ends. A thin biofilm is a biofilm which does not exceed about 10 microns in any given direction.

Bioproduct means a product of a bioconversion which may be an anabolic product or a catabolic product and includes, but is not limited to, primary and secondary metabolites.

A chemical product is one or more chemicals which is desired to be produced. Where the objective of the process is to remove a component from a medium, that component is a substrate and the chemical product is the bioconversion product which may, or may not, have any utility and may be a degradation product. Alternatively, the chemical product is a chemical that has a utility such as an intermediate for use in another chemical process such as, but not limited to, a monomer or feedstock or a chemical that has an application itself as, but not limited to, a foodstuff, nutrient, surfactant, pharmaceutical, insecticide, herbicide, growth promoter or regulator, fuel, solvent, or additive.

Contaminating microorganisms are microorganisms that can foul or compete with the microorganisms for the bioconversion of substrate and may be adventitious or from an up-stream bioconversion process.

Degrade means the conversion of a contaminant to a form that can be removed from the water being treated. For example, metabolic degradation of nitrate generates as the chemical product nitrogen. Whereas, degradation of a metalate anion provides a reduced species that precipitates which may be a metal oxide or the elemental metal, e.g., selenate may be degraded to elemental selenium and borate to boron.

Disparate contaminants are two or more contaminants. Preferred disparate contaminants are those that differ in metabolic pathways for degradation such as reductive metabolic degradation and oxidative metabolic degradation.

A state of essential stasis means that a microorganism population has undergone a substantial cessation of metabolic bioconversion activity but can be revived. The existence of an essential stasis condition can be ascertained by measuring bioconversion activity. The essential stasis condition may be aerobic, anoxic or anaerobic which may or may not be the same as that of normal operating conditions for the microorganism. Where stasis is sought, the temperature is typically in the range of about 0° C. to 25° C., say, 4° C. to 15° C. which may be different from the temperatures used at normal operating conditions.

An exo-network is a community of spaced-apart microorganisms that can bed in the form of individual cells or biofilms that are interconnected by extracellular polymeric substance in the form of strands. The spacing between the microorganisms or biofilms in the exo-network is sufficient to enable the passage of nutrients and substrates there between and is often at least about 0.25, say, at least about 0.5, micron and may be as large as 5 or 10 microns or more.

Exterior skin is an exterior layer of polymer on the biocatalyst that is less open than the major channels in the interior structure of the biocatalyst. A biocatalyst may or may not have a skin. Where a skin is present, it may or may not have surface pores. Where no surface pores are present, fluids diffuse through the skin. Where pores are present, they often have an average diameter of between about 1 and 10 microns.

Fully hydrated means that a biocatalyst is immersed in water at 25° C. until no further expansion of the superficial volume of the biocatalyst is perceived.

The “Hydration Expansion Volume” (HEV) for a biocatalyst is determined by hydrating the biocatalyst in water at 25° C. until the volume of the biocatalyst has stabilized and measuring the superficial volume of the biocatalyst (V_{w}), removing the biocatalyst from water and removing excess water from the exterior, but without drying, and immersing the biocatalyst in ethanol at 25° C. for a time sufficient that the volume of the biocatalyst has stabilized and then measuring the superficial volume of the biocatalyst (Vₛ).

The HEV in volume percent is calculated as the amount of [V_{w}/Nₛ]×100%. To assure dehydration with the ethanol, either a large volume ratio of ethanol to biocatalyst is used or successive immersions of the biocatalyst in fresh ethanol are used. The ethanol is initially dehydrated ethanol.

Irreversibly retained and substantially irreversibly retained mean that the bioactive material is adhering to polymeric structures defining open, porous cavities. Irreversibly retained bioactive material does not include microorganisms located on the exterior surface of a biocatalyst. Bioactive material is irreversibly retained even if the biocatalyst has exterior pores of sufficient size to permit egress of the bioactive material.

Highly hydrophilic polymers are polymers to which water is attracted, i.e., are hydroscopic. Often the polymers exhibit, when cast as a film, a water contact angle of less than about 60°, and sometimes less than about 45°, and in some instances less than about 10°, as measured by the sessile drop method using a 5 microliter drop of pure distilled water.

Highly hydrated means that the volume of the biocatalyst (excluding the volume of the microorganisms) is at least about 90 percent water.

An isolated enzyme is an enzyme removed from a cell and may or may not be in a mixture with other metabolically active or inactive materials.

A matrix is an open, porous, polymeric structure and is an article of manufacture having an interconnected plurality of channels or cavities (herein “major cavities”) defined by polymeric structures, said cavities being between about 5 and 100 microns in the smallest dimension (excluding any microorganisms contained therein) wherein fluid can enter and exit the major cavities from and to the exterior of the matrix. The porous matrix may contain larger and smaller channels or cavities than the major cavities, and may contain channels and cavities not open to the exterior of the matrix. The major cavities, that is, open, interconnected regions of between about 5 or 10 to 70 or 100 microns in the smallest dimension (excluding any microorganism contained therein), have nominal major dimensions of less than about 300, preferably less than about 200, microns, and sometimes a smallest dimension of at least about 10 microns. The term open, porous thus refers to the existence of channels or cavities that are interconnected by openings therebetween.

Metabolic conditions include conditions of temperature, pressure, oxygenation, pH, and nutrients (including micronutrients) and additives required or desired for the microorganisms in the biocatalyst. Nutrients and additives include growth promoters, buffers, antibiotics, vitamins, minerals, nitrogen sources, and sulfur sources and carbon sources where not otherwise provided.

A metalate is an oxyanion, hydroxyl or salt of a metal or semiconductor element.

Oxygenated organic product means a product containing one or more oxygenated organic compounds having 2 to 100, and frequently 2 to 50, carbons and at least one moiety selected from the group consisting of hydroxyl, carbonyl, ether and carboxyl.

Permeable means that a component can enter or exit the major cavities from or to the exterior of the biocatalyst.

A phenotypic change or alternation or phenotypic shift is a change in a microorganism's traits or characteristics from environmental factors and is thus different from a change in the genetic make-up of the microorganism.

Population of microorganisms refers to the number of microorganisms in a given volume and include substantially pure cultures and mixed cultures.

Quiescent means that the aqueous medium in a biocatalyst is still; however, flows of nutrients and substrates and bioproducts can occur through the aqueous medium via diffusion and capillary flow.

Retained solids means that solids are retained in the interior of the biocatalyst. The solids may be retained by any suitable mechanism including, but not limited to, restrained by not being able to pass through pores in the skin of a biocatalyst, by being captured in a biofilm or a polysaccharide structure formed by microorganisms, by being retained in the polymeric structure of the biocatalyst, or by being sterically entangled within the structure of the biocatalyst or the microorganisms.

Smallest dimension means the maximum dimension of the shortest of the maximum dimensions defining the length, width and height of a major cavity. Usually a preponderance of the major cavities in a matrix are substantially width and height symmetrical. Hence the smallest dimension can be approximated by the maximum width of a cavity observed in a two dimensional cross section, e.g., by optical or electronic microscopy.

A solubilized precursor for the polymer is a monomer or prepolymer or the polymer itself that is dissolved or dispersed such that solids cannot be seen by the naked eye and is stable. For instance, a solid can be highly hydrated and be suspended in an aqueous medium even though the solid is not dissolved.

Sorption means any physical or chemical attraction and can be adsorption or absorption and may be relatively weak, e.g., about 10 kilojoules per mole or a chemical interaction with a sorbent. Preferably the sorptive attraction by the sorbent is greater than that between water and the substrate, but not so great that undue energy is required to desorb the substrate. Frequently the sorptive strength is between about 10 and 70, say, 15 and 60, kilojoules per mole. A sorbent is a solid having sorptive capacity for at least one substrate.

A stable population of microorganisms means that the population of microorganisms does not decrease by more than 50 percent nor increase by more than 400 percent.

Substrates are carbon sources, electron donors, electron acceptors and other chemicals that can be metabolized by a microorganism, which chemicals, may or may not provide sustaining value to the microorganisms.

Sugar means carbohydrates having 5 to 12 carbon atoms and includes, but is not limited to, D-glyceraldehyde, L-glyceraldehyde, D-erythrose, L-erythrose, D-threose, L-threose, D-ribose, L-ribose, D-lyxose, L-lyxose, D-allose, L-allose, D-altrose, L-altrose 2-keto-3-deoxy D-gluconate (KDG), D-mannitol, guluronate, mannuronate, mannitol, lyxose, xylitol, D-glucose, L-glucose, D-mannose, L-mannose, D-gluose, L-gluose, D-idose, L-idose, D-galactose, L-galactose, D-xylose, L-xylose, D-arabinose, L-arabinose, D-talose, L-talose, glucuronate, galacturonate, rhamnose, fructooligosaccharide (FOS), galactooligosaccharide (GOS), inulin, mannan oligosaccharide (MOS), oligoalginate, mannuronate, guluronate, alpha-keto acid, or 4-deoxy-L-erythro-hexoselulose uronate (DEHU).

Typical Separation Techniques for chemical products include phase separation for gaseous chemical products, the use of a still, a distillation column, liquid/liquid phase separation, gas stripping, flow-through centrifuge, Karr column for liquid-liquid extraction, mixer-settler, or expanded bed adsorption. Separation and purification steps may proceed by any of a number of approaches combining various methodologies, which may include centrifugation, filtration, reduced pressure evaporation, liquid/liquid phase separation, membranes, distillation, and/or other methodologies recited in this patent application. Principles and details of standard separation and purification steps are known in the art, for example in “Bioseparations Science and Engineering,” Roger G. Harrison et al., Oxford University Press (2003), and Membrane Separations in the Recovery of Biofuels and Biochemicals—An Update Review, Stephen A. Leeper, pp. 99-194, in Separation and Purification Technology, Norman N. Li and Joseph M. Calo, Eds., Marcel Dekker (1992).

The wet weight or wet mass of cells is the mass of cells from which free water has been removed, i.e., are at the point of incipient wetness. All references to mass of cells is calculated on the basis of the wet mass of the cells.

References to organic acids herein shall be deemed to include corresponding salts and esters.

References to biocatalyst dimensions and volumes herein are of fully hydrated biocatalyst unless otherwise stated or clear from the context.

### Biocatalyst

A. Biocatalyst Overview

The biocatalysts of this invention have a polymeric structure (matrix) defining interconnected major cavities, i.e., are open, porous matrices, in which the bioactive material is retained in the interior of the matrices. Where the bioactive material comprises microorganisms, it is believed that the microorganisms and their communities, inter alia, regulate their population. Also, in conjunction with the sensed nature of the microenvironment in the matrices, it is believed that the microorganisms establish a spatial relationship among the members of the community.

The microorganisms that are retained in the matrices often have the ability to form an exo-network. The quiescent nature of the cavities facilitate forming and then maintaining any formed exo-network. A discernable exo-network is not believed essential to achieving phenotypic alterations in the microorganism population such as population modulation and metabolic shift. Where an exo-network develops, often strands of EPS interconnect proximate microorganisms and connect microorganisms to the surface and form the exo-network. In some instances, the microorganisms form thin biofilms and these thin biofilms are encompassed in the exo-network. The biocatalysts have a substantial absence of biofilms in their interiors that are larger than thin biofilms. Hence, any biofilms that may ultimately form in the biocatalysts are relatively thin, e.g., up to about 10, and preferably up to about 2 or 5, microns in thickness, and stable in size. Thus, each thin biofilm is often only a few cells and is connected in an exo-network.

A communication among the microorganisms is believed to occur through emitting chemical agents, including, but not limited to, autoinducers, and communication includes communications for community behavior and for signaling. Often, the preparation of the biocatalysts used in the processes of this invention can result in a population of microorganisms being initially located in the interior of the biocatalyst that is substantially that which would exist at the steady-state level. At these densities of microorganisms in the biocatalysts, community communications are facilitated which are believed to commence during the formation of the biocatalysts, and phenotypic shifts occur to enable the metabolic retention and modulate the population of microorganisms.

Another phenotypic alteration occurring in the biocatalysts, which is believed to be a result of this communication, is a metabolic shift, i.e., the metabolic functions of the community towards reproduction are diminished and the sought bioconversion continues. The population of microorganisms in the biocatalyst may tend to have an old average age due to this shift in the metabolic activity. Older microorganisms also tend to provide a more robust and sustainable performance as compared to younger cells as the older cells have adapted to the operating conditions.

Additional benefits of this communication can be an increase in community-level strength or fitness exhibited by the community in warding off adventitious microorganisms and maintaining strain-type uniformity. In some instances, the microorganisms during use of the biocatalyst may undergo natural selection to cause the strain-type in the community to become heartier or provide another benefit for the survival of the community of microorganisms. In some instances, the communication among the microorganisms may permit the population of microorganisms to exhibit multicellularity or multicellular-like behaviors. Thus the population of microorganisms in a biocatalyst of this invention may have microorganisms adapting to different circumstances but yet working in unison for the benefit of the community.

In some instances the porous matrix may provide modulation of the substrate and nutrients to the microorganisms to effect to optimize metabolic pathways involving substrates that are available, and these pathways may or may not be the primarily used pathways where ample substrate and other nutrients are available. Accordingly, microorganisms in the biocatalysts may exhibit enhanced bioactivity for a primarily used pathway or metabolic activity that is normally repressed.

It is also believed that the microenvironments may promote genetic exchange or horizontal gene transfer. Conjugation or bacterial mating may also be facilitated, including the transfer of plasmids and chromosomal elements. Moreover, where microorganisms lyse, strands of DNA and RNA in the microenvironments are more readily accessible to be taken up by microorganisms in these microenvironments. These phenomena can enhance the functional abilities of the microorganisms.

The biocatalysts exhibit an increased tolerance to toxins. In some instances, communications among microorganisms and the exo-network may facilitate the population establishing defenses against toxins. The community response to the presence of toxins has been observed in the biocatalysts of this invention. For instance, the biocatalysts survive the addition of toxins such as ethanol and sodium hypochlorite and the original bioconversion activity is quickly recovered thus indicating the survival of essentially the entire community.

In summary, due to the microenvironments in the biocatalyst, communication among the microorganisms and the phenotypic alterations undergone by the microorganisms, the biocatalysts provide a number of process-related advantages including, but not limited to,
no solid debris being generated,
the potential for high densities of bioactive material in a bioreactor,
stable population of microorganisms and bioactivity over extended periods of time,
metabolic shift of microorganisms towards production rather than growth and carbon flow shift,
ability of microorganisms to undergo essential stasis for extended durations,
ability to quickly respond to changes in substrate rate of supply and concentration,
attenuation of diauxie,
enhanced control and modulation of pH and redox balances in the microenvironment of the biocatalyst,
greater tolerance to substrate, bioproduct and contaminants,
ability to bioconvert substrate at ultralow concentrations,
ability to use slower growing and less robust microorganisms and increased resistance to competitiveness,
enhanced microorganism strain purity capabilities,
ability to be subjected to in situ antimicrobial treatment,
ability to quickly start a bioreactor since the density of bioactive material required at full operation is contained in the biocatalyst,
ability to contact biocatalyst with gas phase substrate, and
ease of separation of bioproduct from biocatalyst thereby facilitating continuous operations.

If desired, the biocatalysts, where containing microorganisms, may be treated to enhance the formation of the exo-network, and if desired, thin biofilms, prior to use in the metabolic process. However, performance of the porous matrices is not generally dependent upon the extent of exo-network formation, and often bioconversion activities remain relatively unchanged between the time before the microorganisms have attached to the polymeric structure and the time when extensive exo-network structures have been generated.

B. Physical Description of the Porous Matrices

The biocatalysts of this invention comprise a matrix having open, porous interior structure with bioactive material irreversibly retained in at least the major cavities of the matrix.

The matrices may be a self-supporting structure or may be placed on or in a preformed structure such as a film, fiber or hollow fiber, or shaped article. The preformed structure may be constructed of any suitable material including, but not limited to, metal, ceramic, polymer, glass, wood, composite material, natural fiber, stone, and carbon. Where self-supporting, the matrices are often in the form of sheets, cylinders, plural lobal structures such as trilobal extrudates, hollow fibers, or beads which may be spherical, oblong, or free-form. The matrices, whether self-supporting or placed on or in a preformed structure, preferably have a thickness or axial dimension of less than about 5, preferably less than about 2, say, between about 0.01 to 1, centimeters.

The porous matrices may have an isotropic or, preferably, an anisotropic structure with the exterior portion of the cross section having the densest structure. The major cavities, even if an anisotropic structure exists, may be relatively uniform in size throughout the interior of the matrix or the size of the major cavities, and their frequency, may vary over the cross-section of the biocatalyst.

The biocatalyst of this invention has major cavities, that is, open, interconnected regions of between about 5 or 10 to 70 or 100 microns in the smallest dimension (excluding any microorganisms contained therein). For the purposes of ascertaining dimensions, the dimensions of the microorganisms includes any mass in the exo-network. In many instances, the major cavities have nominal major dimensions of less than about 300, preferably less than about 200, microns, and sometimes a smallest dimension of at least about 10 microns. Often the biocatalyst contains smaller channels and cavities which are in open communication with the major cavities. Frequently the smaller channels have a maximum cross-sectional diameter of between about 0.5 to 20, e.g., 1 to 5 or 10, microns. The cumulative volume of major cavities, excluding the volume occupied by microorganisms and mass associated with the microorganisms, to the volume of the biocatalyst is generally in the range of about 40 or 50 to 70 or 99, volume percent. In many instances, the major cavities constitute less than about 70 percent of the volume of the fully catalyst with the remainder constituting the smaller channels and pores. The volume fraction of the biocatalyst that constitute the major cavities can be estimated from its cross-section. The cross section may be observed via any suitable microscopic technique, e.g., scanning electron microscopy and high powered optical microscopy. The total pore volume for the matrices can be estimated from the volumetric measurement of the matrices and the amount and density of polymer, and any other solids used to make the matrices.

The biocatalyst is characterized by having high internal surface areas, often in excess of at least about 1 and sometimes at least about 10, square meter per gram. In some instances, the volume of water that can be held by a fully hydrated biocatalyst (excluding the volume of the microorganisms) is in the range of 90 to 99 or more, percent. Preferably the biocatalyst exhibits a Hydration Expansion Volume (HEV) of at least about 1000, frequently at least about 5000, preferably at least about 20,000, and sometimes between 50,000 and 200,000, percent.

Usually the type of polymer selected and the void volume percent of the matrices are such that the matrices have adequate strength to enable handling, storage and use in a bioconversion process.

The porous matrices may or may not have an exterior skin. Preferably the matrices have an exterior skin to assist in modulating the influx and efflux of components to and from the interior channels of the porous matrix. Also, since the skin is highly hydrophilic, and additional benefit is obtained as contaminating or adventitious microorganisms have difficulties in establishing a strong biofilm on the exterior of the biocatalyst. These contaminating microorganisms are often subject to removal under even low physical forces such as by the flow of fluid around the biocatalysts. Thus, the fouling of the biocatalyst can be substantially eliminated or mitigated by washing or by fluid flows during use.

Where present, the skin typically has pores of an average diameter of between about 1 and 10, preferably 2 to 7, microns in average diameter. The pores may comprise about 1 to 30, say, 2 to 20, percent of the external surface area. The external skin, in addition to providing a barrier to entry of adventitious microorganisms into the interior of the biocatalyst, is preferably relatively smooth to reduce the adhesion of microorganisms to the external side of the skin through physical forces such as fluid flow and contact with other solid surfaces. Often, the skin is substantially devoid of anomalies, other than pores, greater than about 2 or 3 microns. Where a skin is present, its thickness is usually less than about 50, say, between about 1 and 25, microns. It should be understood that the thickness of the skin can be difficult to discern where the porous matrix has an anisotropic structure with the densest structure being at the exterior of the matrix.

A high concentration of isolated enzyme and or density of microorganisms can exist at steady-state operation within the biocatalysts. The combination of the flow channels and the high permeability of the polymeric structure defining the channels enable viable microorganism population throughout the matrix, albeit with a plurality of unique microenvironments and nano-environments. In some instances, when the bioactive material comprises microorganisms, the cell density based upon the volume of the matrices is preferably at least about 100 grams per liter, preferably at least about 200, and often between about 250 and 750, grams per liter.

Polysaccharide-Containing Biocatalysts

In one preferred aspect of the biocatalyst of this invention, it has been found that through incorporating polysaccharide in the interior of the biocatalyst, the viability of the microorganism population can be maintained. Typically polysaccharides are not usable by most microorganisms. Often, the polysaccharide is provided in an amount of at least about 0.1, say, at least about 0.2 to 100, gram per gram of cells retained in the biocatalyst, and sometimes the biocatalyst contains between 25 and 500 grams of polysaccharide per liter of volume of fully hydrated biocatalyst. The polysaccharide particles used in preparing the biocatalysts preferably have a major dimension of less than about 50, preferably less than about 20, often between about 0.1 to 5, microns. The solid polysaccharide particles are preferably granular and often have an aspect ratio of minimum cross-sectional dimension to maximum cross sectional dimension of between about 1:10 to 1:1, say 1:2 to 1:1.

Due to the ability of the polysaccharide to maintain the viability of the microorganisms in the biocatalyst, the storage, handling and processes for use of the biocatalyst can be facilitated. For instance, the biocatalysts can be used in bioconversion processes which are operated in a carbon deficient manner. In metabolic processes where carbon source is added to maintain the microorganisms and not used in the sought bioconversion of substrate to bioproduct, such as in the catabolysis of nitrate, nitrite, and perchlorate anions and the metabolic reduction of metalates, the polysaccharide may serve as the sole source of carbon and thereby eliminate the necessity of adding carbon source, or it may reduce the amount of carbon source added, i.e., permit carbon deficient operation. An advantage is that the bioprocesses can be operated such that the effluent has essentially no COD. The biocatalysts also have enhanced abilities to tolerate disruptions in substrate presence and be able to quickly regain bioconversion activity. Also, the biocatalysts can be remotely manufactured and shipped to the location of use without undue deleterious effect on the bioconversion activity of the biocatalyst. The biocatalysts may be able enter a state of essential stasis for extended durations of time in the absence of supplying substrate and other nutrients to the microbial composites even where excursions in the desired storage conditions such as temperature occur. The bioactivity can be quickly regained in a bioreactor even after extended episodic occurrences of shutdown, feedstock disruption, or feedstock variability. The biocatalysts can be packaged and shipped in sealed barrels, tanks, and the like. The polysaccharide may be from any suitable source including, but not limited to, cellulosic polysaccharides or starches. Polysaccharides are carbohydrates characterized by repeating units linked together by glycosidic bonds and are substantially insoluble in water. Polysaccharides may be homopolysaccharides or heteropolysaccharides and typically have a degree of polymerization of between about 200 and 15,000 or more, preferably between about 200 and 5000. The preferred polysaccharides are those in which about 10, more preferably, at least about 20, percent of the repeating units are amylose (D-glucose units). Most preferably the polysaccharide has at least about 20, more preferably, at least about 30, percent of the repeating units being amylose. The polysaccharides may or may not be functionalized, e.g., with acetate, sulfate, phosphate, pyruvyl cyclic acetal, and the like, but such functionalization should not render the polysaccharide water soluble at temperatures below about 50° C. A preferred class of polysaccharides is starches.

Sources of polysaccharides include naturally occurring and synthetic (e.g., polydextrose) polysaccharides. Various plant based materials providing polysaccharides include but are not limited to woody plant materials providing cellulose and hemicellulose, and wheat, barley, potato, sweet potato, tapioca, corn, maize, cassava, milo, rye and brans typically providing starches.

Solid Sorbent-Containing Biocatalysts

The biocatalysts may contain a solid sorbent. The solid sorbent may be the hydrophilic polymer forming the structure or may be a particulate, i.e., a distinct solid structure regardless of shape) contained in the solid structure. The sorbent may be any suitable solid sorbent for the substrate or nutrients or other chemical influencing the sought metabolic activity such as, but not limited to, co-metabolites, inducers, and promoters or for components that may be adverse to the microorganisms such as, and not in limitation, toxins, phages, bioproducts and by-products. The solid sorbent is typically an adsorbent where the sorption occurs on the surface of the sorbent. The particulate solid sorbents are preferably nano materials having a major dimension less than about 5 microns, preferably, between about 5 nanometers to 3 microns. Where the solid sorbent is composed of polymer, the solid structure may be essentially entirely composed of the polymer or may be a block copolymer or polymeric mixture constituting between about 5 and 90 mass percent of the solid structure (excluding water). Where the solid sorbent is a separate particulate in the biocatalyst, the biocatalyst may comprise between about 5 to 90 mass percent of the mass of the biocatalyst (excluding water and microorganisms but including both the hydrophilic polymer and the particulates). More than one solid sorbent may be used in a biocatalyst. Preferably the solid sorbent is relatively uniformly dispersed throughout the interior of the biocatalyst although the solid sorbent may have a varying distribution within the biocatalyst. Where the distribution varies, the regions with the higher concentration of solid sorbent often are found toward the surface of the biocatalyst.

Where a particulate sorbent is used, the sorbent comprises an organic or inorganic material having the sought sorptive capacity. Examples of solid sorbents include, without limitation, polymeric materials, especially with polar moieties, carbon (including but not limited to activated carbon), silica (including but not limited to fumed silica), silicates, clays, molecular sieves, and the like. The molecular sieves include, but are not limited to zeolites and synthetic crystalline structures containing oxides and phosphates of one or more of silicon, aluminum, titanium, copper, cobalt, vanadium, titanium, chromium, iron, nickel, and the like. The sorptive properties may comprise one or more of physical or chemical or quasi-chemical sorption on the surface of the solid sorbent. Thus, surface area and structure may influence the sorptive properties of some solid sorbents. Frequently the solid sorbents are porous and thus provide high surface area and physical sorptive capabilities. Often the pores in the solid sorbents are in the range of about 0.3 to 2 nanometers in effective diameter.

The solid sorbent may be incorporated into the polymeric structure in any convenient manner, preferably during the preparation of the biocatalyst.

Phosphorescent Biocatalysts

Another preferred aspect of the invention pertains to biocatalysts containing phosphorescent material and photosynthetic microorganisms, i.e., microorganisms that uses light energy in a metabolic process. Preferably the microorganism is an algae, most preferably a microalgae, or cyanobacteria.

The bioactivity of photosynthetic microorganisms can be enhanced to produce expressed bioproduct using broad-based light source such as sunlight. In accordance with the invention, the photosynthetic microorganisms are irreversibly retained in biocatalysts in which the interior of the biocatalyst contains phosphorescent material capable of shifting UV light to light having a wavelength of between about 400 and 800, preferably between about 450 and 650, nm and is capable of exhibiting persistence, with the emission of the light often lasting for at least about 5 seconds. A phosphorescent material is a material that has the ability to be excited by electromagnetic radiation into an excited state, but the stored energy is released gradually. Emissions from phosphorescent materials have persistence, that is, emissions from such materials can last for seconds, minutes or even hours after the excitation source is removed. A luminescent material is a material capable of emitting electromagnetic radiation after being excited into an excited state. Persistence is the time it takes, after discontinuing irradiation, for photoluminescent emissions emanating from a photoluminescent object to decrease to the threshold detectability.

The persistence of the radiation enables the microorganisms to be cycled in and out of a region of the culture liquid exposed to the light source and still be productive. With longer persistence durations, the photosynthetic microorganisms can continue photo-bioconversion in the absence of or reduction in light intensity. The ability of the biocatalysts to maintain photosynthetic activity over extended periods of time, often at least about 30 days, and in some instances for at least one year, the cost of the phosphorescent materials is well offset by the increased production, reduced footprint of the bioreactor, and facilitated bioproduct recovery.

The biocatalyst, being highly hydrated is a significant distributor of light radiation to photosynthetic microorganisms retained in the interior of the biocatalyst and also serves to protect the microorganism from photorespiration. The solid debris in the culture liquid (an aqueous solution comprising nutrients for metabolic processes) can be materially reduced, if not essentially eliminated, due to the microorganisms being irreversibly retained in the biocatalyst. Thus the turbidity is reduced and a given light intensity can thus be found at a greater depth in the culture liquid. These advantages provided by the biocatalysts of this invention can be realized in any photosynthetic process regardless of whether or not a phosphorescent material is used.

Examples of phosphorescent materials include, but are not limited to, phosphorescent materials are metal sulfide phosphors such as ZnCdS:Cu:Al, ZnCdS:Ag:Al, ZnS:Ag:Al, ZnS:Cu:Al as described in U.S. Pat. No. 3,595,804 and metal sulfides that are co-activated with rare earth elements such as those describe in U.S. Pat. No. 3,957,678. Phosphors that are higher in luminous intensity and longer in luminous persistence than the metal sulfide pigments include compositions comprising a host material that is generally an alkaline earth aluminate, or an alkaline earth silicate. The host materials generally comprise Europium as an activator and often comprise one or more co-activators such as elements of the Lanthanide series (e.g. lanthanum, cerium, praseodymium, neodymium, samarium, gadolinium, terbium, dysprosium, holmium, erbium, thulium, ytterbium, and lutetium), tin, manganese, yttrium, or bismuth. Examples of such phosphors are described in U.S. Pat. No. 5,424,006.

High emission intensity and persistence phosphorescent materials can be alkaline earth aluminate oxides having the formula MOₘAl₂O₃:Eu²⁺, R³⁺ wherein m is a number ranging from 1.6 to about 2.2, M is an alkaline earth metal (strontium, calcium or barium), Eu²⁺ is an activator, and R is one or more trivalent rare earth materials of the lanthanide series (e.g. lanthanum, cerium, praseodymium, neodymium, samarium, gadolinium, terbium, dysprosium, holmium, erbium, thulium, ytterbium, lutetium), yttrium or bismuth co-activators. Examples of such phosphors are described in U.S. Pat. No. 6,117,362. Phosphorescent materials also include alkaline earth aluminate oxides having the formula Mₖ Al₂O₄:2xEu²⁺, 2yR³⁺ wherein k=1−2×−2y, x is a number ranging from about 0.0001 to about 0.05, y is a number ranging from about x to 3x, M is an alkaline earth metal (strontium, calcium or barium), Eu²⁺ is an activator, and R is one or more trivalent rare earth materials (e.g. lanthanum, cerium, praseodymium, neodymium, samarium, gadolinium, terbium, dysprosium, holmium, erbium, thulium, ytterbium, lutetium), yttrium or bismuth co-activators. See U.S. Pat. No. 6,267,911B1.

Phosphorescent materials also include those in which a portion of the Al³⁺ in the host matrix is replaced with divalent ions such as Mg²⁺ or Zn²⁺ and those in which the alkaline earth metal ion (M²⁺) is replaced with a monovalent alkali metal ion such as Li⁺, Na⁺, K⁺, Cs⁺ or Rb⁺ such as described in U.S. Pat. Nos. 6,117,362 and 6,267,911B1.

High intensity and high persistence silicates have been disclosed in U.S. Pat. No. 5,839,718, such as Sr.BaO.Mg.MO.SiGe:Eu:Ln wherein M is beryllium, zinc or cadmium and Ln is chosen from the group consisting of the rare earth materials, the group 3A elements, scandium, titanium, vanadium, chromium, manganese, yttrium, zirconium, niobium, molybdenum, hafnium, tantalum, tungsten, indium, thallium, phosphorous, arsenic, antimony, bismuth, tin, and lead. Particularly useful are dysprosium, neodymium, thulium, tin, indium, and bismuth. X in these compounds is at least one halide atom.

Other phosphorescent materials include alkaline earth aluminates of the formula MO.Al₂O₃.B₂O₃:R wherein M is a combination of more than one alkaline earth metal (strontium, calcium or barium or combinations thereof) and R is a combination of Eu²⁺ activator, and at least one trivalent rare earth material co-activator, (e.g. lanthanum, cerium, praseodymium, neodymium, samarium, gadolinium, terbium, dysprosium, holmium, erbium, thulium, ytterbium, lutetium), bismuth or manganese. Examples of such phosphors can be found in U.S. Pat. No. 5,885,483. Alkaline earth aluminates of the type MAl₂O₄, which are described in U.S. Pat. No. 5,424,006, may also find application as may phosphorescent materials comprising a donor system and an acceptor system such as described in U.S. Pat. No. 6,953,536 B2.

As can be appreciated, many other phosphors can find application. See, for instance, Yen and Weber, Inorganic Phosphors: Compositions, Preparation and Optical Properties, CRC Press, 2004.

The phosphorescent material may be a discrete particle or may be a particle having a coating to facilitate incorporation and retention in the polymer forming the matrix. The particles may be of any suitable shape. Generally the maximum dimension of the of the particles is less than about 1 millimeter, preferably less than about 0.1 millimeter. The particles may be nanoparticles.

The persistence time exhibited by the phosphorescent materials can range from a short duration, e.g., about 5 to 10 seconds, to as much as 10 or 20 hours or more and will be dependent upon the phosphorescent material used. Preferred phosphorescent materials exhibit a persistence of at least about one minute. The intensity of the emitted radiation from the polymer of the matrices will, in part, depend upon the concentration of the phosphorescent material in the polymer and the nature of the phosphorescent material. Typically the phosphorescent material is provided in an amount of at least about 0.1, say, between 0.2 and 5 or 10, mass percent of the polymer (non-hydrated) in the biocatalyst. One or more phosphorescent materials may be used in the biocatalyst. Where more than one phosphorescent material are used, the combination may be selected to provide one or more of wave shifting from different light wavelengths contained in the band width of the radiation source and providing differing persistence times. In preferred embodiments the phosphorescent materials are in the form of nanoparticles, e.g., having a major dimension of between about 10 nm and 10 μm. In some instances, it may be desired to coat the phosphorescent materials with a compatibilizing agent to facilitate incorporation of the phosphorescent material within the polymer. Compatibilizing agents include, but are not limited to, molecules having one or more of hydroxyl, thiol, silyl, carboxyl, or phosphoryl groups.

C. Methods for Making Biocatalysts

The components, including bioactive materials, used to make the biocatalysts and the process conditions used for the preparation of the biocatalysts are not critical to the broad aspects of this invention and may vary widely as is well understood in the art once understanding the principles described above. In any event, the components and process conditions for making the biocatalysts with the irreversibly, metabolically retained microorganisms should not adversely affect the microorganisms.

The biocatalysts may be prepared from a liquid medium containing the bioactive material and solubilized precursor for the hydrophilic polymer which may be one or more of a polymerizable or solidifiable component or a solid that is fusible or bondable to form the matrix. Aqueous media are most often used due to the compatibility of most microorganisms and enzymes with water. However, with bioactive materials that tolerate other liquids, such liquids can be used to make all or a portion of the liquid medium. Examples of such other liquids include, but are not limited to liquid hydrocarbons, peroxygenated liquids, liquid carboxy-containing compounds, and the like. Mixed liquid media can also be used to prepare the biocatalyst. The mixed media may comprise miscible or immiscible liquid phases. For instance, the bioactive material may be suspended in a dispersed, aqueous phase and the polymerizable or solidifiable component may be contained in a continuous solvent phase.

The liquid medium used to prepare the biocatalyst may contain more than one type of microorganism, especially where the microorganisms do not significantly compete for the same substrate, and may contain one or more isolated enzymes or functional additives such as polysaccharide, solid sorbent and phosphorescent materials, as described above. Preferably, the biocatalysts contain a single type of microorganism. The concentration of the microorganisms in the liquid medium used to make the biocatalysts should at least be about 60 grams per liter. As discussed above, the concentration of microorganisms should preferably approximate the sought density of microorganisms in the biocatalyst. The relative amounts of microorganism and polymeric material in forming the biocatalyst can vary widely. The growth of the population of microorganisms post formation of the biocatalyst is contemplated as well as the potential for damage to some of the population of microorganisms during the biocatalyst-forming process. Nevertheless, higher microorganism concentrations are generally preferred, e.g., at least about 100 grams per liter, preferably at least about 200, and often between about 250 and 750, grams per liter of the liquid medium used to make the biocatalysts.

Any suitable process may be used to solidify or polymerize the polymeric material or to adhere or fuse particles to form the open, porous polymeric matrix with bioactive material irreversibly retained therein. The conditions of suitable processes should not unduly adversely affect the bioactive material. As bioactive materials differ in tolerance to temperatures, pressures and the presence of other chemicals, some matrix-forming processes may be more advantageous for one type of bioactive material than for another type of bioactive material.

Preferably the polymeric matrix is formed from solidification of a high molecular weight material, by polymerization or by cross-linking of prepolymer in manner that a population of microorganisms is provided in the interior of the biocatalyst as it is being formed. Exemplary processes include solution polymerization, slurry polymerization (characterized by having two or more initial phases), and solidification by cooling or removal of solvent.

The biocatalysts may be formed in situ in the liquid medium by subjecting the medium to solidification conditions (such as cooling or evaporation) or adding a component to cause a polymerization or cross-linking or agglomeration of solids to occur to form a solid structure such as a catalyst, cross-linking agent or coagulating agent. Alternatively, the liquid medium may be extruded into a solution containing a solidification agent such as a catalyst, cross-linking or coagulating agent or coated onto a substrate and then the composite subjected to conditions to form the solid biocatalyst.

Polymeric materials used to make the biocatalysts may have an organic or inorganic backbone but have sufficient hydrophilic moieties to provide a highly hydrophilic polymer which when incorporated into the matrices exhibits sufficient water sorption properties to provide the sought Hydration Expansion Volume of the biocatalyst. Polymeric materials are also intended to include high molecular weight substances such as waxes (whether or not prepared by a polymerization process), oligomers and the like so long as they form biocatalysts that remain solid under the conditions of the bioconversion process intended for their use and have sufficient hydrophilic properties that the Hydration Expansion Volume can be achieved. As stated above, it is not essential that polymeric materials become cross-linked or further polymerized in forming the polymeric matrix.

Examples of polymeric materials include homopolymers and copolymers which may or may not be cross-linked and include condensation and addition polymers that provide high hydrophilicity and enable the Hydration Expansion Volumes to be obtained. The polymer may be a homopolymer or a copolymer, say, of a hydrophilic moiety and a more hydrophobic moiety. The molecular weight and molecular weight distribution are preferably selected to provide the combination of hydrophilicity and strength as is known in the art. The polymers may be functionalized with hydrophilic moieties to enhance hydrophilicity. Examples of hydrophilic moieties include, but are not limited to hydroxyl, alkoxyl, acyl, carboxyl, amido, and oxyanions of one or more of titanium, molybdenum, phosphorus, sulfur and nitrogen such as phosphates, phosphonates, sulfates, sulfonates, and nitrates, and the hydrophilic moieties may be further substituted with hydrophilic moieties such as hydroxyalkoxides, acetylacetonate, and the like. Typically the polymers contain carbonyl and hydroxyl groups, especially at some adjacent hydrophilic moieties such as glycol moieties. In some instances, the backbone of the polymer contains ether oxygens to enhance hydrophilicity. In some instances, the atomic ratio of oxygen to carbon in the polymer is between about 0.3:1 to 5:1.

Polymers which may find use in forming the matrices include functionalized or non-functionalized polyacrylamides, polyvinyl alcohols, polyetherketones, polyurethanes, polycarbonates, polysulfones, polysulfides, polysilicones, olefinic polymers such as polyethylene, polypropylene, polybutadiene, rubbers, and polystyrene, nylons, polythyloxazyoline, polyethylene glycol, polysaccharides such as sodium alginate, carageenan, agar, hyaluronic acid, chondroitin sulfate, dextran, dextran sulfate, heparin, heparin sulfate, heparan sulfate, chitosan, gellan gum, xanthan gum, guar gum, water soluble cellulose derivatives and carrageenan, and proteins such as gelatin, collagen and albumin, which may be polymers, prepolymers or oligomers, and polymers and copolymers from the following monomers, oligomers and prepolymers:

monomethacrylates such as polyethylene glycol monomethacrylate, polypropylene glycol monomethacrylate, polypropylene glycol monomethacrylate, methoxydiethylene glycol methacrylate, methoxypolyethylene glycol methacrylate, methacryloyloxyethyl hydrogen phthalate, methacryloyloxyethyl hydrogen succinate, 3-chloro-2-hydroxypropyl methacrylate, stearyl methacrylate, 2-hydroxy methacrylate, and ethyl methacrylate; monoacrylates such as 2-hydroxyethyl acrylate, 2-hydroxypropyl acrylate, isobutyl acrylate, t-butyl acrylate, isooctyl acrylate, lauryl acrylate, stearyl acrylate, isobornyl acrylate, cyclohexyl acrylate, methoxytriethylene glycol acrylate, 2-ethoxyethyl acrylate, tetrahydrofurfuryl acrylate, phenoxyethyl acrylate, nonylphenoxypolyethylene glycol acrylate, nonylphenoxypolypropylene glycol acrylate, silicon-modified acrylate, polypropylene glycol monoacrylate, phenoxyethyl acrylate, phenoxydiethylene glycol acrylate, phenoxypolyethylene glycol acrylate, methoxypolyethylene glycol acrylate, acryloyloxyethyl hydrogen succinate, and lauryl acrylate;
dimethacrylates such as 1,3-butylene glycol dimethacrylate, 1,4-butanediol dimethacrylate, ethylene glycol dimethacrylate, diethylene glycol dimethacrylate, triethylene glycol dimethacrylate, polyethylene glycol dimethacrylate, butylene glycol dimethacrylate, hexanediol dimethacrylate, neopentyl glycol dimethacrylate, polyprene glycol dimethacrylate, 2-hydroxy-1,3-dimethacryloxypropane, 2,2-bis-4-methacryloxyethoxyphenylpropane, 3,2-bis-4-methacryloxydiethoxyphenylpropane, and 2,2-bis-4-methacryloxypolyethoxyphenylpropane;
diacrylates such as ethoxylated neopentyl glycol diacrylate, polyethylene glycol diacrylate, 1,6-hexanediol diacrylate, neopentyl glycol diacrylate, tripropylene glycol diacrylate, polypropylene glycol diacrylate, 2,2-bis-4-acryloxyethoxyphenylpropane, 2-hydroxy-1-acryloxy-3-methacryloxypropane; trimethacrylates such as trimethylolpropane trimethacrylate; triacrylates such as trimethylolpropane triacrylate, pentaerythritol triacrylate, trimethylolpropane EO-added triacrylate, glycerol PO-added triacrylate, and ethoxylated trimethylolpropane triacrylate; tetraacrylates such as pentaerythritol tetraacrylate, ethoxylated pentaerythritol tetraacrylate, propoxylated pentaerythritol tetraacrylate, and ditrimethylolpropane tetraacrylate;
urethane acrylates such as urethane acrylate, urethane dimethyl acrylate, and urethane trimethyl acrylate;
amino-containing moieties such as 2-aminoethyl acrylate, 2-aminoethyl methacrylate, aminoethyl methacrylate, dimethyl aminoethyl methacrylate, monomethyl aminoethyl methacrylate, t-butylaminoethylmethacrylate, p-aminostyrene, o-aminostyrene, 2-amino-4-vinyltoluene, dimethylaminoethyl acrylate, diethylaminoethyl acrylate, piperidinoethyl ethyl acrylate, piperidinoethyl methacrylate, morpholinoethyl acrylate, morpholinoethyl methacrylate, 2-vinyl pyridine, 3-vinyl pyridine, 2-ethyl-5-vinyl pyridine, dimethylaminopropylethyl acrylate, dimethylaminopropylethyl methacrylate, 2-vinyl pyrrolidone, 3-vinyl pyrrolidone, dimethylaminoethyl vinyl ether, dimethylaminoethyl vinyl sulfide, diethylaminoethyl vinyl ether, 2-pyrrolidinoethyl acrylate, 2-pyrrolidinoethyl methacrylate,
and other monomers such as acrylamide, acrylic acid, and dimethylacrylamide.

Not all the above listed polymers will be useful by themselves, but may be required to be functionalized or used to form a co-polymer with a highly hydrophilic polymer.

Cross linking agents, accelerators, polymerization catalysts, and other polymerization additives may be employed such as triethanolamine, triethylamine, ethanolamine, N-methyl diethanolamine, N,N-dimethyl benzylamine, dibenzyl amino, N-benzyl ethanolamine, N-isopropyl benzylamino, tetramethyl ethylenediamine, potassium persulfate, tetramethyl ethylenediamine, lysine, ornithine, histidine, arginine, N-vinyl pyrrolidinone, 2-vinyl pyridine, 1-vinyl imidazole, 9-vinyl carbazone, acrylic acid, and 2-allyl-2-methyl-1,3-cyclopentane dione. For polyvinyl alcohol polymers and copolymers, boric acid and phosphoric acid may be used in the preparation of polymeric matrices. As stated above, the amount of cross-linking agent may need to be limited to assure that the matrices retain high hydrophilicity and the ability to have a high Hydration Expansion Volume. The selection of the polymer and cross-linking agents and other additives to make porous matrices having the physical properties set forth above is within the level of the artisan in the art of water soluble and highly hydrophilic polymer synthesis.

The biocatalysts may be formed in the presence of other additives which may serve to enhance structural integrity or provide a beneficial activity for the microorganism such as attracting or sequestering components, providing nutrients, and the like. Additives can also be used to provide, for instance, a suitable density to be suspended in the aqueous medium rather than tending to float or sink in the broth. Typical additives include, but are not limited to, starch, glycogen, cellulose, lignin, chitin, collagen, keratin, clay, alumina, aluminosilicates, silica, aluminum phosphate, diatomaceous earth, carbon, polymer, polysaccharide and the like. These additives can be in the form of solids when the polymeric matrices are formed, and if so, are often in the range of about 0.01 to 100 microns in major dimension.

If desired, where the biocatalyst contains microorganisms, they may be subjected to stress as is known in the art. Stress may be one or more of starvation, chemical or physical conditions. Chemical stresses include toxins, antimicrobial agents, and inhibitory concentrations of compounds. Physical stresses include light intensity, UV light, temperature, mechanical agitation, pressure or compression, and desiccation or osmotic pressure. The stress may produce regulated biological reactions that protect the microorganisms from shock and the stress may allow the hardier microorganisms to survive while the weaker cells die.

### Bioactive Material

The bioactive material is one or more of isolated enzymes and microorganisms. In the processes of this invention, at least one biocatalyst contains microorganisms, and preferably at least two biocatalysts contain microorganisms. In another aspect, the biocatalysts can contain, in addition to the microorganisms, one or more extracellular enzymes in the interior of the biocatalyst to cause a catalytic change to a component which may be substrate or other nutrients, or a bioproduct or by-product or co-product of the microorganisms, or may be a toxin, phage or the like.

Examples of enzymes include, but are not limited to, one or more of oxidorectases, transferases, hydrolases, lyases, isomerases, and ligases. The enzymes may cause one or more metabolic conversions. For instance, an enzyme may metabolize a component in the feed such that it can be bioconverted, or more easily be bioconverted, by the microorganisms in the biocatalyst. An enzyme may be used to metabolize a metabolite of the microorganism either to provide a sought bioproduct. An enzyme may be used to metabolize a component in the feed or a co-metabolite from the microorganism that may be adverse to the microorganism into a metabolite that is less adverse to the microorganism. If desired, two or more different enzymes can be used to effect a series of metabolic conversions on a component in the feed or a metabolite from the microorganism.

Representative enzymes include, without limitation: cellulase, cellobiohydrolase (e.g., CBHI, CBHII), alcohol dehydrogenase (A, B, and C), acetaldehyde dehydrogenase, amylase, alpha amylase, glucoamylase, beta glucanase, beta glucosidase, invertase, endoglucanase (e.g., EGI, EGII, EGIII), lactase, hemicellulase, pectinase, hydrogenase, pullulanase, phytase, a hydrolase, a lipase, polysaccharase, ligninase, Accellerase® 1000, Accellerase® 1500, Accellerase® DUET, Accellerase® TRIO, or Cellic CTec2 enzymes, phosphoglucose isomerase, inositol-1-phosphate synthase, inositol monophosphatase, myo-inositol dehydrogenase, myo-inosose-2-dehydratase, inositol 2-dehydrogenase, deoxy-D-gluconate isomerase, kinase, 5-dehydro-2-deoxygluconokinase, deoxyphophogluconate aldolase, 3-hydroxy acid dehydrogenase, isomerase, topoisomerase, dehydratase, monosaccharide dehydrogenase, aldolase, phosphatase, a protease, DNase, alginate lyase, laminarinase, endoglucanase, L-butanediol dehydrogenase, acetoin reductase, 3-hydroxyacyl-CoA dehydrogenase, or cis-aconitate decarboxylase. The enzymes include those described by Heinzelman et al. (2009) PNAS 106: 5610-5615, herein incorporated by reference in its entirety.

The enzymes may be bound to the precursor for the hydrophilic polymer of the biocatalyst prior to the formation of the biocatalyst or may be introduced during the preparation of the biocatalyst, e.g., by addition to the liquid medium for forming the biocatalyst. There are many methods that would be known to one of skill in the art for providing enzymes or fragments thereof, or nucleic acids, onto a solid support. Some examples of such methods include, e.g., electrostatic droplet generation, electrochemical means, via adsorption, via covalent binding, via cross-linking, via a chemical reaction or process. Various methods are described in Methods in Enzymology, Immobilized Enzymes and Cells, Part C. 1987. Academic Press. Edited by S. P. Colowick and N. O. Kaplan. Volume 136; Immobilization of Enzymes and Cells. 1997. Humana Press. Edited by G. F. Bickerstaff. Series: Methods in Biotechnology, Edited by J. M. Walker; DiCosimo, R., McAuliffe, J., Poulose, A. J. Bohlmann, G. 2012. Industrial use of immobilized enzymes. Chem. Soc. Rev.; and Immobilized Enzymes: Methods and Applications. Wilhelm Tischer and Frank Wedekind, Topics in Current Chemistry, Vol. 200. Page 95-126.

Typically extracellular enzymes bond or adhere to solid surfaces, such as the hydrophilic polymer, solid additives, cell walls and extracellular polymeric substance. Hence, the enzymes can be substantially irreversibly retained in the interior of the biocatalyst. Due to the structure of the biocatalysts of this invention, the microorganisms and the enzymes can be in close proximity and thus effective, cooperative bioconversions can be obtained. The association of the enzymes with the interior surfaces of the biocatalyst typically increases the resistance of the enzyme or enzymes to denaturation due to changes in temperature, pH, or other factors related to thermal or operational stability of the enzymes. Also, by being retained in the biocatalyst, the use of the enzyme in a bioreactor is facilitated and undesirable post-reactions can be mitigated.

Where the bioactive material comprises microorganisms, the microorganisms may be unicellular or may be multicellular that behaves as a single cell microorganism such as filamentous growth microorganisms and budding growth microorganisms. Often the cells of multicellular microorganisms have the capability to exist singularly. The microorganisms can be of any type, including, but not limited to, those microorganisms that are aerobes, anaerobes, facultative anaerobes, heterotrophs, autotrophs, photoautotrophs, photoheterotrophs, chemoautotrophs, and/or chemoheterotrophs. The cellular activity, including cell can be growing aerobic, microaerophilic, or anaerobic. The cells can be in any phase of growth, including lag (or conduction), exponential, transition, stationary, death, dormant, vegetative, sporulating, etc. The one or more microorganisms be a psychrophile (optimal growth at −10° C. to 25° C.), a mesophile (optimal growth at 20-50° C.), a thermophile (optimal growth 45° C. to 80° C.), or a hyperthermophile (optimal growth at 80° C. to 100° C.). The one or more microorganisms can be a gram-negative or gram-positive bacterium. A bacterium can be a cocci (spherical), bacilli (rod-like), or spirilla (spiral-shaped; e.g., vibrios or comma bacteria). The microorganisms can be phenotypically and genotypically diverse.

The microorganisms can be a wild-type (naturally occurring) microorganism or a recombinant (including, but not limited to genetically engineered microorganisms) microorganism. A recombinant microorganism can comprise one or more heterologous nucleic acid sequences (e.g., genes). One or more genes can be introduced into a microorganism used in the methods, compositions, or kits described herein, e.g., by homologous recombination. One or more genes can be introduction into a microorganism with, e.g., a vector. The one or more microorganisms can comprise one or more vectors. A vector can be an autonomously replicating vector, i.e., a vector that exists as an extra-chromosomal entity, the replication of which is independent of chromosomal replication, e.g., a linear or closed circular plasmid, an extra-chromosomal element, a mini-chromosome, or an artificial chromosome. The vector can contain a means for self-replication. The vector can, when introduced into a host cell, integrate into the genome of the host cell and replicate together with the one or more chromosomes into which it has been integrated. Such a vector can comprise specific sequences that can allow recombination into a particular, desired site of the host chromosome. A vector system can comprise a single vector or plasmid, two or more vectors or plasmids, which together contain the total DNA to be introduced into the genome of the host cell, or a transposon. The choice of the vector will typically depend on the compatibility of the vector with the host cell into which the vector is to be introduced. The vector can include a reporter gene, such as a green fluorescent protein (GFP), which can be either fused in frame to one or more of the encoded polypeptides, or expressed separately. The vector can also include a selection marker such as an antibiotic resistance gene that can be used for selection of suitable transformants. Means of genetically manipulating organisms are described, e.g., Current Protocols in Molecular Biology, last updated Jul. 25, 2011, Wiley, Print ISSN: 1934-3639. In some embodiments, one or more genes involved in byproduct formation are deleted in a microorganism. In some embodiments, one or more genes involved in byproduct formation are not deleted. Nucleic acid introduced into a microorganism can be codon-optimized for the microorganism. A gene can be modified (e.g., mutated) to increase the activity of the resulting gene product (e.g., enzyme). Sought properties in wild-type or genetically modified microorganisms can often be enhanced through a natural modification process, or self-engineering process, involving multigenerational selective harvesting to obtain strain improvements such as microorganisms that exhibit enhanced properties such as robustness in an environment or bioactivity. See, for instance, Ben-Jacob, et al., Self-engineering capabilities of bacteria, J. R. Soc. Interface 2006, 3, doi: 10.1098/rsif.2005.0089, 22 Feb. 2006.

The selected microorganism to be used in a biocatalyst can be targeted to the sought activity. The biocatalysts thus often contain substantially pure strain types of microorganisms and, because of the targeting, enable high bioactivity to be achieved and provide a stable population of the microorganism in the biocatalyst.

Representative microorganisms for making biocatalysts of this invention include, without limitation, those set forth in United States published patent application nos. 2011/0072714, especially paragraph 0122; 2010/0279354, especially paragraphs 0083 through 0089; 2011/0185017, especially paragraph 0046; 2009/0155873; especially paragraph 0093; and 20060063217, especially paragraphs 0030 and 0031, and those setforth in Appendix A.

Photosynthetic microorganisms include bacteria, algae, and molds having biocatalytic activity activated by light radiation. Examples of photosynthetic microorganisms for higher oxygenated organic compound production include, but are not limited to alga such as Bacillariophyceae strains, Chlorophyceae, Cyanophyceae, Xanthophyceaei, Chrysophyceae, Chlorella (e.g., Chlorella protothecoides), Crypthecodinium, Schizocytrium, Nannochloropsis, Ulkenia, Dunaliella, Cyclotella, Navicula, Nitzschia, Cyclotella, Phaeodactylum, and Thaustochytrids; yeasts such as Rhodotorula, Saccharomyces, and Apiotrichum strains; and fungi species such as the Mortierella strain. Genetically enhanced photoautotrophic cyanobacteria, algae, and other photoautotrophic organisms have been adapted to bioconvert carbohydrates internal to the microorganism directly to ethanol, butanol, pentanol and other higher alcohols and other biofuels. For example, genetically modified cyanobacteria having constructs comprising DNA fragments encoding pyruvate decarboxylase (pdc) and alcohol dehydrogenase (adh) enzymes are described in U.S. Pat. No. 6,699,696. Cyanobacteria are photosynthetic bacteria which use light, inorganic elements, water, and a carbon source, generally carbon dioxide, to metabolize and grow. The production of ethanol using genetically engineered cyanobacteria has also been described in PCT Published Patent Application WO 2007/084477.

### Process Configurations

The bioconversion processes and apparatus of this invention use at least two biocatalysts, at least one of which has microorganisms that are irreversibly retained in a porous matrix. If a free suspension of microorganisms is used in addition to a biocatalyst, then the biocatalyst containing other microorganisms should have an exterior surface that is substantially impermeable to the microorganisms in free suspension. Preferably, where at least one type of microorganism is freely suspended in the aqueous medium, the process conditions are such that undue fouling of the surface of the biocatalyst does not occur.

The biocatalyst configurations or modes can be selected to provide one or more results as described below
Plural substrate consumption mode: In the plural substrate consumption mode, at least two substrates are supplied to the aqueous medium, at least one of which is not substantially converted by at least one biocatalyst but is bioconverted by another biocatalyst. The chemical products of the bioconversion by the biocatalysts may be the same or different. In one aspect, the biocatalysts convert the substrates to the same chemical product. An example of a plural substrate consumption mode is where C₅ and C₆ sugars are supplied to an aqueous medium and one biocatalyst converts the C₅ sugar to ethanol and byproducts and another biocatalyst converts the C₆ sugars to ethanol and byproducts. Another example of a plural substrate consumption mode is where the feed is an aqueous stream containing a plurality of contaminants and different biocatalysts degrade different contaminants.
Sequential consumption mode: In the sequential consumption mode, at least one substrate supplied to the aqueous medium is bioconverted by at least one biocatalyst to an intermediate chemical product and at least one other biocatalyst converts the intermediate chemical product to chemical product. An example of a sequential consumption mode is where sugar is supplied to an aqueous medium and one biocatalyst converts the sugar to ethanol and acetate, an intermediate chemical product, and another biocatalyst converts the acetate to ethanol.
Combination plural and sequential consumption mode: The combination plural and sequential mode is a combination of the plural and sequential modes where at least two substrates are provided to the aqueous medium and at least one biocatalyst converts one of the substrates to a first intermediate chemical product and at least one other biocatalyst converts another of the substrates to a second intermediate chemical product and then a third biocatalyst requires at least the first intermediate chemical product and the second intermediate chemical product to at least one chemical product. In this mode, the bioconversion by the third biocatalyst may require both the first and second intermediate chemicals to produce the chemical product or may convert each of the first and second intermediate chemicals to different or the same chemical product.
Inducing mode: In this mode, one biocatalyst produces a chemical product that is an inducer, promoter or co-metabolite which is used by another biocatalyst in the aqueous medium to bioconvert a substrate.
Byproduct elimination mode: In the byproduct consumption mode, a first biocatalyst produces a chemical product and an intermediate chemical and at least one other biocatalyst bioconverts the intermediate chemical to another intermediate chemical which is not used to make a chemical product. In this mode, the intermediate chemical produced using the first biocatalyst may be deleterious to the first biocatalyst or not otherwise desired in the aqueous medium due to environmental or further processing considerations.

The processes of this invention also include the use of a single biocatalyst structure that contains regions with different microorganisms, herein referred to as a “layered” biocatalyst. By “layered” it is meant that substrate for microorganisms in at least one region is generated or passes through another region. The microorganisms in the different regions may operate in any of the above modes. However, since the microorganisms are metabolically retained in the regions, competition can be constrained and a stable population of each of the microorganisms can be maintained. The layered biocatalysts can be made by any suitable process. For instance, a biocatalyst can be prepared and then that biocatalyst added to the solution for making an encompassing biocatalyst. Alternatively, where the microorganisms that are aerobic and microorganisms that are anaerobic are used, the biocatalyst may be made with a mixture of the microorganisms with ultimately each specie predominating in regions of the biocatalyst providing acceptable environments.

The selection of the biocatalysts is typically based upon the sought biocatalyst configuration mode, the substrate and the sought chemical product. Moreover, the biocatalysts selected should be capable of being able to operate under common bioconversion conditions such as temperature and nutrients as well as having tolerance to contaminants in the feedstock providing the substrate and tolerance for the concentrations of substrate, chemical product and intermediate chemicals that may exist in the common aqueous medium. Moreover, while it is typically desired to have biocatalysts that prefer aerobic, anaerobic or anoxic conditions used with biocatalysts that prefer similar aerobic or anaerobic conditions, the biocatalysts often provide microenvironments that provide the desired oxygen concentration. Thus, aerobic biocatalysts sometimes are able to be used with anaerobic biocatalysts in a common aqueous medium.

The preferred aspects of this invention permit the relative amounts of each of the biocatalysts to be maintained in desired ranges. The capability is especially beneficial in continuous bioconversion processes to avoid a build-up of one or more of substrate or intermediate chemical.

### General Process

The apparatus of this invention broadly pertain to bioreactors for the bioconversion of at least one substrate to at least one chemical product comprising: a vessel defining an interior volume; an aqueous medium contained in at least a portion of the interior volume of the vessel; and at least two biocatalysts distributed within the aqueous medium. The apparatus and the processes may be adapted for continuous, semi-continuous or batch bioconversion. The vessel may be a sealed vessel or may be an open vessel such as a pond or open tank. The material of the vessel can be any suitable material that is substantially impervious to the aqueous medium and may be metal, ceramic, polymeric, clay, or the like. The vessel may be rigid or flexible as a bag or polymeric film.

Bioreactors may be of any suitable design. Exemplary designs include, but are not limited to, bubble column reactors, stirred reactors, packed bed reactors, trickle bed reactors, fluidized bed reactors, plug flow (tubular) reactors, and membrane (biofilm) reactors. In conducting photosynthetic bioconversions, the reactors may be designed to permit the transfer of photo energy.

Where two or more biocatalysts are used, both all biocatalysts may be freely mobile, at least one mobile and at least one other fixed, or all may be fixed. Where more than one biocatalyst is fixed, it may be interspersed with or separate from at least one other fixed biocatalyst. Where at least two biocatalysts are freely mobile, the biocatalysts may be interspersed in the aqueous medium or separated by fluid permeable barriers. The fluid permeable barriers may be, e.g., screens, draft tubes, and looped reactors where a common aqueous medium is contacted with the biocatalysts. More than one reactor vessel may be used. For instance, reactor vessels may be in parallel or in sequential flow series.

The aqueous medium in the bioreactor may not be subjected to any external mixing force such as in a batch reactor. Alternatively, the aqueous medium may be agitated by the flow of feeds to the reactor, by pumped recirculation of aqueous medium or by mechanical agitation. For photo-activated biocatalyst processes, aqueous medium containing biocatalysts may be sprayed to enhance exposure to light and provide agitation or the biocatalysts may form, or be supported to form, high surface area exposure to the light source with the aqueous medium flowing over the biocatalysts. Preferably, the agitation is not unduly deleterious to the biocatalyst.

The bioreactors may be designed to enable energy to be supplied to the aqueous medium and biocatalysts. The energy may be one or more of heat, electrical and radiation. For photo-activated biocatalytic processes, the radiation may be natural or artificial.

The metabolic processes using the biocatalysts may be conducted in any suitable manner employing metabolic conditions sufficient for the biocatalyst to convert the substrate to the sought bioproduct. Metabolic conditions include conditions of temperature, pressure, oxygenation, pH, and nutrients (including micronutrients) and additives required or desired for the microorganisms in the biocatalyst. Due to the microenvironments and phenotypic alterations associated with the biocatalysts of this invention, often a broader range of metabolic conditions can be effectively used than those suitable for planktonic microorganisms.

The metabolic processes using the biocatalysts of this invention provide sufficient water to the biocatalyst to maintain the biocatalyst hydrated. The bioconversion processes may involve direct contact with gas containing substrate or in contact with a liquid medium, often an aqueous medium. Water for this aqueous medium may be provided from any suitable source including, but not limited to, tap water, demineralized water, distilled water, and process or waste water streams. The aqueous medium can contain nutrients and additives such as co-metabolites, potentiators, enhancers, inducers growth promoters, buffers, antibiotics, vitamins, minerals, nitrogen sources, and sulfur sources as is known in the art. If desired, an antifoam agent may be used in the aqueous medium. In some instances, where additives are desired or required for the metabolic process, the biocatalysts of this invention exhibit at least equivalent bioconversion activity at a lesser concentration of such additives as compared to a planktonic, free-suspension system, all else being substantially the same.

The processes may be conducted with all carbon requirements being provided in the aqueous medium or on a carbon source deficient basis. Where operating in a carbon source deficiency, the aqueous medium often provides at least about 50, frequently at least about 75, say, 80 to less than 100, mass percent on a carbon basis of the carbon nutrient. In some instances polysaccharide is included in the biocatalyst where carbon source deficiency operations are anticipated. The carbon source deficiency may occur intermittently or continuously during the metabolic process.

The bioconversion processes may be optimized to achieve one or more objectives. For instance, the processes may be designed to provide high conversions of substrate to bioproduct or may be designed to balance capital and energy costs against conversion to bioproduct. As the biocatalysts are highly hydrated, generally their density is close to that of water. Accordingly, with fluidized bed reactor designs using an aqueous feed stream, energy consumption is lower than that where higher density supports are used. In some instances where the metabolic processes generate a gas, e.g., in the conversion of sugars to alkanols or in the bioconversion of nitrate anion to nitrogen gas, gas can accumulate in the biocatalyst to increase buoyancy. This accumulated gas can reduce the energy consumption for a fluid bed operation and can facilitate the use of other bioreactor designs such as jet loop bioreactors.

The bioproduct may be recovered from the aqueous medium in any suitable manner including the Typical Separation Techniques.

Examples of anabolic or catabolic processes suitable to be practiced by the processes of this invention include, but are not limited to:
Syngas, i.e., gas containing carbon monoxide and optionally hydrogen, for conversion to oxygenated organic product and hydrocarbons. In typical prior art processes for the conversion of syngas to oxygenated organic product, a limiting factor on productivity is the mass transfer of carbon monoxide and hydrogen from the gas phase into the liquid phase of the aqueous medium. By using the biocatalysts of this invention for syngas bioconversion, mass transfer can be enhanced.
Carbon dioxide-containing gases for conversion to oxygenated organic product and hydrocarbons. The anabolic conversion may be effected by algae, cyanobacteria, or other photo activated microorganisms, e.g., to produce alcohols, biodiesel, and like. Other bioconversion processes using carbon dioxide to produce bioproducts include those to make organic acids and esters and diacids and diesters such as succinic acid and lactic acid.
Combustion gases, e.g., from the disposal of solid wastes or generation of energy, where the substrate comprises contaminants sought to be removed from the gases such as oxygenated halides, sulfoxy moieties, nitrogen oxides, heavy metal compounds and the like.
Industrial process waste gases containing, for instance, volatile organic compounds; solvents such as chlorine containing solvents, ketones, aldehydes, peroxygenates, and the like; ammonia or volatile amines; mercaptans and other sulfur containing compounds; nitrogen oxides; and the like. The industrial process waste gases may be air-based, such as exhaust from painting operations, or maybe devoid of air such as purge or waste gases. The ability to subject these substrates to catabolic degradation can often eliminate the necessity for a thermal oxidation unit operation resulting in both capital and energy savings as often natural gas or other fuel is required to maintain temperature for the thermal oxidation unit.
Natural gas (including, but not limited to, gas recovered by underground fracturing processes, i.e., frac gas) wherein the substrate for catabolic processing may be one or more of oxygenates, such as nitrogen oxides, sulfur oxides; perchlorates; sulfides, ammonia; mercaptans; and the like.
Nitrates, perchlorates, taste and odor compounds, organics, chlorinated hydrocarbons, and the like removal from the water. The source of the water may be from a water treatment facility, ground sources, surface sources, municipal waste processing, and industrial waste water. The water stream may be derived from other bioconversion processes where substrate is not fully consumed, such as in corn ethanol processes.
Carbohydrate, including, but not limited to cellulose, hemicellulose, starches, and sugars for conversion to oxygenated organic product and hydrocarbons.
Oxyanions, hydroxyls or soluble salts of sulfur, phosphorus, selenium, tungsten, molybdenum, bismuth, strontium, cadmium, chromium, titanium, nickel, iron, zinc, copper, arsenic, vanadium, uranium, radium, manganese, germanium, indium, antimony mercury, and rare earth metals for removal from water by bioconversion and sequestration.

Often the microorganisms retained in the biocatalysts exhibit a tolerance to toxins and antimicrobial agents. This enhanced tolerance can be particularly attractive to control or eliminate populations of competitive microorganisms in the aqueous medium by the continuous or intermittent addition of antimicrobial agent to the aqueous medium. Preferably the antimicrobial agent is a sterilizing agent, and most preferably is an oxidizing agent. These preferred sterilizing agents are relatively inexpensive and include hydrogen peroxide, peracetic acid, aldehydes (especially glutaraldehyde and o-phthalaldehyde), ozone, and hypochlorite. Examples of bacteriostatic agents include nitroimidazoles, nitrofurans, rifampin, chloramphenicol, tetracyclines, aminoglycosides, macrolides and lincosamides. Bacteriostatic agents can be preferred in instances where inhibition of growth of the population of the contaminating microorganism is sufficient to maintain the desired operation of the process.

### Biofuel and Bioproduct Embodiments and Configurations

The processes of this invention can be used to produce biofuels and bioproducts from substrates.

Substrates can be natural or xenobiotic substances in an organism (plant or animal) or can be obtained from other sources. Hence, substrates include, but are not limited to, those that can be, or can be derived from, plant, animal or fossil fuel sources, or can be produced by a chemical or industrial process. The biocatalysts generate metabolites as a result of anabolic or catabolic activity and the metabolites may be primary or secondary metabolites. The processes of this invention can be used to produce any type of anabolic metabolite.

Bioproducts may be one or more of aliphatic compounds and aromatic compounds including but not limited to hydrocarbons of up to 44 or 50 carbons, and hydrocarbons substituted with one or more of hydroxyl, acyl, carboxyl, amine, amide, halo, nitro, sulfonyl, and phosphino moieties, and hydrocarbons containing one or more hetero atoms including but not limited to, nitrogen, sulfur, oxygen, and phosphorus atoms. Examples of organic products as end products from metabolic processes are those listed in United States published patent application no. 2010/0279354 A1, especially as set forth in paragraphs 0129 through 0149. See also, United States published patent application no. 2011/0165639 A1. Other bioproducts include p-toluate, terephthalate, terephthalic acid, aniline, putrescine, cyclohexanone, adipate, hexamethylenediamine (HMDA), 6-aminocaproic acid, malate, acrylate, apidipic acid, methacrylic acid, 3-hydroxypropionic acid (3HP), succinate, butadiene, propylene, caprolactam, fatty alcohols, fatty acids, glycerates, acrylic acid, acrylate esters, methacrylic acid, methacrylic acids, fucoidan, muconate, iodine, chlorophyll, carotenoid, calcium, magnesium, iron, sodium, potassium, and phosphate. The bioproduct may be a chemical that provides a biological activity with respect to a plant or animal or human. The biological activity can be one or more of a number of different activities such as antiviral, antibiotic, depressant, stimulant, growth promoters, hormone, insulin, reproductive, attractant, repellant, biocide, and the like. Examples of antibiotics include, but are not limited to, aminoglycosides (e.g., amikacin, gentamicin, kanamycin, neomycin, netilmicin, tobramycin, paromomycin); ansamycins (e.g., geldanamycin, herbimycin); carbacephem (loracarbef); carbapenems (e.g., ertapenem, doripenem, imipenem/cilastatin, meropenem); cephalosporins (first generation, e.g., cefadroxil, cefazolin, cefalotin, cefalexin); cephalosporins (second generation, e.g., cefaclor, cefamandole, cefoxitin, cefprozil, cefuroxime); cephalosporins (third generation, e.g., cefixime, cefdinir, cefditoren, cefoperazone, cefotaxime, cefpodoxime, ceftazidime, ceftibuten, ceftizoxime, ceftriaxone); cephalosporins (fourth generation, e.g., cefepime); cephalosporins (fifth generation, e.g., ceftobiprole); glycopeptides (e.g., teicoplanin, vancomycin, telavancin); lincosamides (e.g., clindamycin, lincomycin); macrolides (e.g., azithromycin, clarithromycin, dirithromycin, erythromycin, roxithromycin, troleandomycin, telithromycin spectinomycin); monobactams (e.g., aztreonam); nitrofurans (e.g., furazolidone, nitrofurantoin); penicillins (e.g., amoxicillin, ampicillin, azlocillin, carbenicillin, cloxacillin, dicloxacillin, flucloxacillin, mezlocillin, methicillin, nafcillin, oxacillin, penicillin G, penicillin V, piperacillin, temocillin, ticarcillin); penicillin combinations (e.g., amoxicillin/clavulanate, ampicillin/sulbactam, piperacillin/tazobactam, ticarcillin/clavulanate); polypeptides (e.g., bacitracin, colistin, polymyxin B); quinolones (e.g., ciprofloxacin, enoxacin, gatifloxacin, levofloxacin, lomefloxacin, moxifloxacin, nalidixic acid, norfloxacin, ofloxacin, trovafloxacin, grepafloxacin, sparfloxacin, temafloxacin); sulfonamides (e.g., mafenide; sulfonamidochrysoidine, sulfacetamide, sulfadiazine, silver sulfadiazine, sulfamethizole, sulfamethoxazole, sulfanilimide, sulfasalazine, sulfisoxazole, trimethoprim, trimethoprim-Sulfamethoxazole (Co-trimoxazole) (TMP-SMX); tetracyclines (e.g., demeclocycline, doxycycline, minocycline, oxytetracycline, tetracycline); drugs against mycobacteria (e.g., clofazimine, dapsone, capreomycin, cycloserine, ethambutol, ethionamide, isoniazid, pyrazinamide, rifampin, rifabutin, rifapentine, streptomycin) and others (e.g., arsphenamine, chloramphenicol, fosfomycin, fusidic acid, linezolid, metronidazole, mupirocin, platensimycin, luinupristin/dalfopristin, rifaximin, thiamphenicol, tinidazole).

### Water Treatment Embodiments and Configurations

Water treatment can be a plural substrate consumption mode of the processes of this invention to degrade or otherwise remove contaminants. Examples of contaminants in water that can be treated using the processes of this invention include organic and inorganic compounds capable of being metabolically reduced or oxidized. The nature of the contaminants is not broadly critical to this invention other than the water to be treated contains disparate contaminants. Hence, the processes of this invention can be adapted to treat a wide variety of water compositions from a wide variety of sources such as ground water, surface water, municipal waste water and industrial water streams. The processes of this invention can often be beneficially employed where the water may contain contaminants that are not sought to be treated such a salt and naturally occurring microorganisms, e.g., where the water is derived from a ground or surface water source. An additional benefit that can be realized by the processes of this invention is that by maintaining the microorganisms substantially irreversibly retained in the biocatalyst, microbial contamination of the treated water by these microorganisms can be substantially avoided.

Examples of contaminants in water include, but are not limited to, hydrocarbons, such as aliphatic and aromatic hydrocarbons of 1 to 50 or more carbons, including alkanes, alkenes, and alkynes, and aromatics such as benzene, toluene and xylene; ethers, ketones, aldehydes, alcohols, carboxylic acids and esters of 1 to 50 or more carbons; halogenated hydrocarbons such as brominated and chlorinated hydrocarbons including perchloroethylene, dichloroethylene, vinyl chloride, trichloroethane, trichloroethylene, methylene chloride, chloroform, carbon tetrachloride and polychlorinated biphenyls (PCB's), and soluble metal and semi-metal compounds including nitrates, nitrites, sulfates, sulfites, phosphates, phosphites, and other metalates. The processes can also be used to substantially reduce contaminants that may be present in the water at very low concentrations. These contaminants include products of algal blooms such as methylisoboreal (MIB) and geosmin, 1,4-dioxane, and N-nitrosodimethylamine (NDMA). It is within the skill of the art to identify microorganisms useful for the degradation of a contaminant, many of which are described above.

Bioproducts may be degradation products especially where contaminants are removed from water. Such degradation bioproducts include, but are not limited to, carbon dioxide, carbon monoxide, hydrogen, carbonyl sulfide, hydrogen sulfide, water, and salts such as carbonate, bicarbonate, sulfide, sulfite, sulfate, phosphate, phosphite, chloride, bromide, iodide, and borate salts of ammonium, or group 1 to 16 (IUPAC) metals such as sodium, potassium, manganese, magnesium, calcium, barium, iron, copper, cobalt, tin, selenium, radium, uranium, bismuth, cadmium, mercury, molybdenum and tungsten.

### DRAWINGS

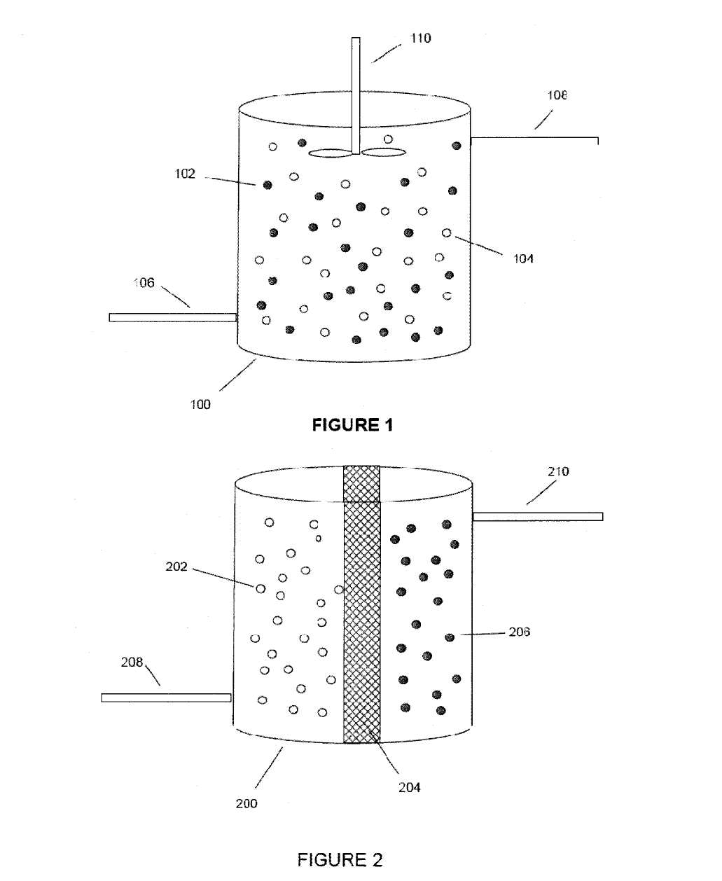

The drawings are provided to facilitate understanding the broad aspects of the invention and are not in limitation of the invention. FIG. 1 is a schematic representation of a bioreactor having vessel 100 with an aqueous medium having therein beads of a first biocatalyst 102 and a second biocatalyst 104 freely dispersed in the aqueous medium. Substrate and nutrients are provided via line 106, and the flow facilitates agitation of the aqueous medium and the dispersion of the biocatalysts therein. Line 108 serves to remove aqueous medium for chemical product recovery. Agitator 110 serves to mix the aqueous medium and facilitate maintaining the biocatalyst dispersed. Alternatively, biocatalyst 102 and biocatalyst 104 may be interspersed and held in a fixed bed, e.g., with screens, and substrate and nutrients would be fed via line 108 and aqueous medium would flow downwardly over the bed and be withdrawn via line 106.

FIG. 2 is a schematic representation of a bioreactor having vessel 200 containing aqueous medium. Vessel 200 contains three biocatalysts. For the purposes of this illustration, beads of biocatalyst 202 are separated from beads of biocatalyst 206 by a fixed structure of third biocatalyst on a three dimensional mesh support 204. The composite biocatalyst and mesh support 204 is permeable to aqueous medium but is not permeable to biocatalysts 202 and 206. Substrate and nutrients are provided to the bioreactor via line 208, and aqueous medium is withdrawn via line 210 for product recovery.

### APPENDIX A

Representative microorganisms include, without limitation, Acetobacter sp., Acetobacter aceti, Achromobacter, Acidiphilium, Acidovorax delafieldi P4-1, Acinetobacter sp. (A. calcoaceticus), Actinomadura, Actinoplanes, Actinomycetes, Aeropyrum pernix, Agrobacterium sp., Alcaligenes sp. (A. dentrificans), Alloiococcus otitis, Ancylobacter aquaticus, Ananas comosus (M), Arthrobacter sp., Arthrobacter sulfurous, Arthrobacter sp. (A. protophormiae), Aspergillus sp., Aspergillus niger, Aspergillus oryze, Aspergillus melleus, Aspergillus pulverulentus, Aspergillus saitoi, Aspergillus sojea, Aspergillus usamii, Bacillus alcalophilus, Bacillus amyloliquefaciens, Bacillus brevis, Bacillus circulans, Bacillus clausii, Bacillus cereus, Bacillus lentus, Bacillus licheniformis, Bacillus macerans, Bacillus stearothermophilus, Bacillus subtilis, Beijerinckia sp., Bifidobacterium, Brevibacterium sp. HL4, Brettanomyces sp., Brevibacillus brevis, Burkholderia cepacia, Campylobacter jejuni, Candida sp., Candida cylindracea, Candida rugosa, Carboxydothermus (Carboxydothermus hydrogenoformans), Carica papaya (L), Cellulosimicrobium, Cephalosporium, Chaetomium erraticum, Chaetomium gracile, Chlorella sp., Citrobacter, Clostridium sp., Clostridium butyricum, Clostridium acetobutylicum, Clostridium kluyveri, Clostridium carboxidivorans, Clostridium thermocellum, Cornynebacterium sp. strain m15, Corynebacterium (glutamicum), Corynebacterium efficiens, Deinococcus radiophilus, Dekkera, Dekkera bruxellensis, Escherichia coli, Enterobacter sp., Enterococcus, Enterococcus faecium, Enterococcus gallinarium, Enterococcus faecalis, Erwinia sp., Erwinia chrysanthemi, Gliconobacter, Gluconacetobacter sp., Hansenula sp., Haloarcula, Humicola insolens, Humicola nsolens, Kitasatospora setae, Klebsiella sp., Klebsiella oxytoca, Klebsiella pneumonia, Kluyveromyces sp., Kluyveromyces fragilis, Kluyveromyces lactis, Kocuria, Lactlactis, Lactobacillus sp., Lactobacillus fermentum, Lactobacillus sake, Lactococcus, Lactococcus lactis, Leuconostoc, Methylosinus trichosporum OB3b, Methylosporovibrio methanica 812, Methanothrix sp. Methanosarcina sp., Methanomonas sp., Methylocystis, Methanospirilium, Methanolobus siciliae, Methanogenium organophilum, Methanobacerium sp., Methanobacterium bryantii, Methanococcus sp., Methanomicrobium sp., Methanoplanus sp., Methanosphaera sp., Methanolobus sp., Methanoculleus sp., Methanosaeta sp., Methanopyrus sp., Methanocorpusculum sp., Methanosarcina, Methylococcus sp., Methylomonas sp., Methylosinus sp., Microbacterium imperiale, Micrococcus sp., Micrococcus lysodeikticus, Microlunatus, Moorella (e.g., Moorella (Clostridium) thermoacetica), Moraxella sp. (strain B), Morganella, Mucor javanicus, Mycobacterium sp. strain GP1, Myrothecium, Neptunomonas naphthovorans, Nitrobacter, Nitrosomonas (Nitrosomonas europea), Nitzchia sp., Nocardia sp., Pachysolen sp., Pantoea, Papaya carica, Pediococcus sp., Pediococcus halophilus, Penicillium, Penicillium camemberti, Penicillium citrinum, Penicillium emersonii, Penicillium roqueforti, Penicillum lilactinum, Penicillum multicolor, Phanerochaete chrysoporium, Pichia sp., Pichia stipitis, Paracoccus pantotrophus, Pleurotus ostreatus, Propionibacterium sp., Proteus, Pseudomonas (P. pavonaceae, Pseudomonas ADP, P. stutzeri, P. putida, Pseudomonas Strain PS1, P. cepacia G4, P. medocina KR, P. picketti PK01, P. vesicularis, P. paucimobilis, Pseudomonas sp. DLC-P11, P. mendocina, P. chichhori, strain IST 103), Pseudomonas fluorescens, Pseudomonas denitrificans, Pyrococcus, Pyrococcus furiosus, Pyrococcus horikoshii, Ralstonia sp., Rhizobium, Rhizomucor miehei, Rhizomucor pusillus Lindt, Rhizopus, Rhizopus delemar, Rhizopus japonicus, Rhizopus niveus, Rhizopus oryzae, Rhizopus oligosporus, Rhodococcus, (R. erythropolis, R. rhodochrous NCIMB 13064), Salmonella, Saccharomyces sp., Saccharomyces cerevisiae, Schizochytriu sp., Sclerotina libertina, Serratia sp., Shigella, Sphingobacterium multivorum, Sphingobium (Sphingbium chlorophenolicum), Sphingomonas (S. yanoikuyae, S. sp. RW1), Streptococcus, Streptococcus thermophilus Y-I, Streptomyces, Streptomyces griseus, Streptomyces lividans, Streptomyces murinus, Streptomyces rubiginosus, Streptomyces violaceoruber, Streptoverticillium mobaraense, Synechococcus sp., Synechocystis sp., Tetragenococcus, Thermus, Thiosphaera pantotropha, Trametes, Trametes versicolor, Trichoderma, Trichoderma longibrachiatum, Trichoderma reesei, Trichoderma viride, Trichosporon sp., Trichosporon penicillatum, Vibrio alginolyticus, Xanthomonas, Xanthobacter sp. (X. autotrophicus GJ10, X. flavus), yeast, Yarrow lipolytica, Zygosaccharomyces rouxii, Zymomonas sp., Zymomonus mobilis, Geobacter sulfurreducens, Geobacter lovleyi, Geobacter metallireducens, Bacteroides succinogens, Butyrivibrio fibrisolvens, Clostridium cellobioparum, Ruminococcus albus, Ruminococcus flavefaciens, Eubacterium cellulosolvens, Clostridium cellulosolvens, Clostridium cellulovorans, Clostridium thermocellum, Bacteroides cellulosolvens, and Acetivibrio cellulolyticus Gliricidia sp., Albizia sp., or Parthenium sp. Cupriavidus basilensis, Cupriavidus campinensis, Cupriavidus gilardi, Cupriavidus laharsis, Cupriavidus metallidurans, Cupriavidus oxalaticus, Cupriavidus pauculus, Cupriavidus pinatubonensis, Cupriavidus respiraculi, Cupriavidus taiwanensis, Oligotropha carboxidovorans, Thiobacillus sp., Thiobacillus denitrificans, Thiobacillus thioxidans, Thiobacillus ferrooxidans, Thiobacillus concretivorus, Acidithiobacillus albertensis, Acidithiobacillus caldus, Acidithiobacillus cuprithermicus, Rhodopseudomonas, Rhodopseudomonas palustris, Rhodobacter sphaeroides, Rhodopseudomonas capsulate, Rhodopseudomonas acidophila, Rhodopseudomonas viridis, Desulfotomaculum, Desulfotomaculum acetoxidans, Desulfotomaculum kuznetsovii, Desulfotomaculum nigrificans, Desulfotomaculum reducens, Desulfotomaculum carboxydivorans, Methanosarcina barkeri, Methanosarcina acetivorans, Moorella thermoacetica, Carboxydothermus hydrogenoformans, Rhodospirillum rubrum, Acetobacterium woodii, Butyribacterium methylotrophicum, Clostridium autoethanogenum, Clostridium ljungdahlii, Eubacterium limosum, Oxobacter pfennigii, Peptostreptococcus productus, Rhodopseudomonas palustris P4, Rubrivivax gelatinosus, Citrobacter sp Y19, Methanosarcina acetivorans C2A, Methanosarcina barkeri, Desulfosporosinus orientis, Desulfovibrio desulfuricans, Desulfovibrio vulgaris, Moorella thermoautotrophica, Carboxydibrachium pacificus, Carboxydocella thermoautotrophica, Thermincola carboxydiphila, Thermolithobacter carboxydivorans, Thermosinus carboxydivorans, Methanothermobacter thermoautotrophicus, Desulfotomaculum carboxydivorans, Desulfotomaculum kuznetsovii, Desulfotomaculum nigrificans, Desulfotomaculum thermobenzoicum subsp. thermosyntrophicum, Syntrophobacter fumaroxidans, Clostridium acidurici, Desulfovibrio africanus, C. pasteurianum, C. pasteurianum DSM 525, Paenibacillus polymyxa, Acanthoceras, Acanthococcus, Acaryochloris, Achnanthes, Achnanthidium, Actinastrum, Actinochloris, Actinocyclus, Actinotaenium, Amphichrysis, Amphidinium, Amphikrikos, Amphipleura, Amphiprora, Amphithrix, Amphora, Anabaena, Anabaenopsis, Aneumastus, Ankistrodesmus, Ankyra, Anomoeoneis, Apatococcus, Aphanizomenon, Aphanocapsa, Aphanochaete, Aphanothece, Apiocystis, Apistonema, Arthrodesmus, Artherospira, Ascochloris, Asterionella, Asterococcus, Audouinella, Aulacoseira, Bacillaria, Balbiania, Bambusina, Bangia, Basichlamys, Batrachospermum, Binuclearia, Bitrichia, Blidingia, Botrdiopsis, Botrydium, Botryococcus, Botryosphaerella, Brachiomonas, Brachysira, Brachytrichia, Brebissonia, Bulbochaete, Bumilleria, Bumilleriopsis, Caloneis, Calothrix, Campylodiscus, Capsosiphon, Carteria, Catena, Cavinula, Centritractus, Centronella, Ceratium, Chaetoceros, Chaetochloris, Chaetomorpha, Chaetonella, Chaetonema, Chaetopeltis, Chaetophora, Chaetosphaeridium, Chamaesiphon, Chara, Characiochloris, Characiopsis, Characium, Charales, Chilomonas, Chlainomonas, Chlamydoblepharis, Chlamydocapsa, Chlamydomonas, Chlamydomonopsis, Chlamydomyxa, Chlamydonephris, Chlorangiella, Chlorangiopsis, Chlorella, Chlorobotrys, Chlorobrachis, Chlorochytrium, Chlorococcum, Chlorogloea, Chlorogloeopsis, Chlorogonium, Chlorolobion, Chloromonas, Chlorophysema, Chlorophyta, Chlorosaccus, Chlorosarcina, Choricystis, Chromophyton, Chromulina, Chroococcidiopsis, Chroococcus, Chroodactylon, Chroomonas, Chroothece, Chrysamoeba, Chrysapsis, Chrysidiastrum, Chrysocapsa, Chrysocapsella, Chrysochaete, Chrysochromulina, Chrysococcus, Chrysocrinus, Chrysolepidomonas, Chrysolykos, Chrysonebula, Chrysophyta, Chrysopyxis, Chrysosaccus, Chrysophaerella, Chrysostephanosphaera, Clodophora, Clastidium, Closteriopsis, Closterium, Coccomyxa, Cocconeis, Coelastrella, Coelastrum, Coelosphaerium, Coenochloris, Coenococcus, Coenocystis, Colacium, Coleochaete, Collodictyon, Compsogonopsis, Compsopogon, Conjugatophyta, Conochaete, Coronastrum, Cosmarium, Cosmioneis, Cosmocladium, Crateriportula, Craticula, Crinalium, Crucigenia, Crucigeniella, Cryptoaulax, Cryptomonas, Cryptophyta, Ctenophora, Cyanodictyon, Cyanonephron, Cyanophora, Cyanophyta, Cyanothece, Cyanothomonas, Cyclonexis, Cyclostephanos, Cyclotella, Cylindrocapsa, Cylindrocystis, Cylindrospermum, Cylindrotheca, Cymatopleura, Cymbella, Cymbellonitzschia, Cystodinium Dactylococcopsis, Debarya, Denticula, Dermatochrysis, Dermocarpa, Dermocarpella, Desmatractum, Desmidium, Desmococcus, Desmonema, Desmosiphon, Diacanthos, Diacronema, Diadesmis, Diatoma, Diatomella, Dicellula, Dichothrix, Dichotomococcus, Dicranochaete, Dictyochloris, Dictyococcus, Dictyosphaerium, Didymocystis, Didymogenes, Didymosphenia, Dilabifilum, Dimorphococcus, Dinobryon, Dinococcus, Diplochloris, Diploneis, Diplostauron, Distrionella, Docidium, Draparnaldia, Dunaliella, Dysmorphococcus, Ecballocystis, Elakatothrix, Ellerbeckia, Encyonema, Enteromorpha, Entocladia, Entomoneis, Entophysalis, Epichrysis, Epipyxis, Epithemia, Eremosphaera, Euastropsis, Euastrum, Eucapsis, Eucocconeis, Eudorina, Euglena, Euglenophyta, Eunotia, Eustigmatophyta, Eutreptia, Fallacia, Fischerella, Fragilaria, Fragilariforma, Franceia, Frustulia, Curcilla, Geminella, Genicularia, Glaucocystis, Glaucophyta, Glenodiniopsis, Glenodinium, Gloeocapsa, Gloeochaete, Gloeochrysis, Gloeococcus, Gloeocystis, Gloeodendron, Gloeomonas, Gloeoplax, Gloeothece, Gloeotila, Gloeotrichia, Gloiodictyon, Golenkinia, Golenkiniopsis, Gomontia, Gomphocymbella, Gomphonema, Gomphosphaeria, Gonatozygon, Gongrosia, Gongrosira, Goniochloris, Gonium, Gonyostomum, Granulochloris, Granulocystopsis, Groenbladia, Gymnodinium, Gymnozyga, Gyrosigma, Haematococcus, Hafniomonas, Hallassia, Hammatoidea, Hannaea, Hantzschia, Hapalosiphon, Haplotaenium, Haptophyta, Haslea, Hemidinium, Hemitoma, Heribaudiella, Heteromastix, Heterothrix, Hibberdia, Hildenbrandia, Hillea, Holopedium, Homoeothrix, Hormanthonema, Hormotila, Hyalobrachion, Hyalocardium, Hyalodiscus, Hyalogonium, Hyalotheca, Hydrianum, Hydrococcus, Hydrocoleum, Hydrocoryne, Hydrodictyon, Hydrosera, Hydrurus, Hyella, Hymenomonas, Isthmochloron, Johannesbaptistia, Juranyiella, Karayevia, Kathablepharis, Katodinium, Kephyrion, Keratococcus, Kirchneriella, Klebsormidium, Kolbesia, Koliella, Komarekia, Korshikoviella, Kraskella, Lagerheimia, Lagynion, Lamprothamnium, Lemanea, Lepocinclis, Leptosira, Lobococcus, Lobocystis, Lobomonas, Luticola, Lyngbya, Malleochloris, Mallomonas, Mantoniella, Marssoniella, Martyana, Mastigocoleus, Gastogloia, Melosira, Merismopedia, Mesostigma, Mesotaenium, Micractinium, Micrasterias, Microchaete, Microcoleus, Microcystis, Microglena, Micromonas, Microspora, Microthamnion, Mischococcus, Monochrysis, Monodus, Monomastix, Monoraphidium, Monostroma, Mougeotia, Mougeotiopsis, Myochloris, Myromecia, Myxosarcina, Naegeliella, Nannochloris, Nautococcus, Navicula, Neglectella, Neidium, Nephroclamys, Nephrocytium, Nephrodiella, Nephroselmis, Netrium, Nitella, Nitellopsis, Nitzschia, Nodularia, Nostoc, Ochromonas, Oedogonium, Oligochaetophora, Onychonema, Oocardium, Oocystis, Opephora, Ophiocytium, Orthoseira, Oscillatoria, Oxyneis, Pachycladella, Palmella, Palmodictyon, Pnadorina, Pannus, Paralia, Pascherina, Paulschulzia, Pediastrum, Pedinella, Pedinomonas, Pedinopera, Pelagodictyon, Penium, Peranema, Peridiniopsis, Peridinium, Peronia, Petroneis, Phacotus, Phacus, Phaeaster, Phaeodermatium, Phaeophyta, Phaeosphaera, Phaeothamnion, Phormidium, Phycopeltis, Phyllariochloris, Phyllocardium, Phyllomitas, Pinnularia, Pitophora, Placoneis, Planctonema, Planktosphaeria, Planothidium, Plectonema, Pleodorina, Pleurastrum, Pleurocapsa, Pleurocladia, Pleurodiscus, Pleurosigma, Pleurosira, Pleurotaenium, Pocillomonas, Podohedra, Polyblepharides, Polychaetophora, Polyedriella, Polyedriopsis, Polygoniochloris, Polyepidomonas, Polytaenia, Polytoma, Polytomella, Porphyridium, Posteriochromonas, Prasinochloris, Prasinocladus, Prasinophyta, Prasiola, Prochlorphyta, Prochlorothrix, Protoderma, Protosiphon, Provasoliella, Prymnesium, Psammodictyon, Psammothidium, Pseudanabaena, Pseudenoclonium, Psuedocarteria, Pseudochate, Pseudocharacium, Pseudococcomyxa, Pseudodictyosphaerium, Pseudokephyrion, Pseudoncobyrsa, Pseudoquadrigula, Pseudosphaerocystis, Pseudostaurastrum, Pseudostaurosira, Pseudotetrastrum, Pteromonas, Punctastruata, Pyramichlamys, Pyramimonas, Pyrrophyta, Quadrichloris, Quadricoccus, Quadrigula, Radiococcus, Radiofilum, Raphidiopsis, Raphidocelis, Raphidonema, Raphidophyta, Peimeria, Rhabdoderma, Rhabdomonas, Rhizoclonium, Rhodomonas, Rhodophyta, Rhoicosphenia, Rhopalodia, Rivularia, Rosenvingiella, Rossithidium, Roya, Scenedesmus, Scherffelia, Schizochlamydella, Schizochlamys, Schizomeris, Schizothrix, Schroederia, Scolioneis, Scotiella, Scotiellopsis, Scourfieldia, Scytonema, Selenastrum, Selenochloris, Sellaphora, Semiorbis, Siderocelis, Diderocystopsis, Dimonsenia, Siphononema, Sirocladium, Sirogonium, Skeletonema, Sorastrum, Spermatozopsis, Sphaerellocystis, Sphaerellopsis, Sphaerodinium, Sphaeroplea, Sphaerozosma, Spiniferomonas, Spirogyra, Spirotaenia, Spirulina, Spondylomorum, Spondylosium, Sporotetras, Spumella, Staurastrum, Stauerodesmus, Stauroneis, Staurosira, Staurosirella, Stenopterobia, Stephanocostis, Stephanodiscus, Stephanoporos, Stephanosphaera, Stichococcus, Stichogloea, Stigeoclonium, Stigonema, Stipitococcus, Stokesiella, Strombomonas, Stylochrysalis, Stylodinium, Styloyxis, Stylosphaeridium, Surirella, Sykidion, Symploca, Synechococcus, Synechocystis, Synedra, Synochromonas, Synura, Tabellaria, Tabularia, Teilingia, Temnogametum, Tetmemorus, Tetrachlorella, Tetracyclus, Tetradesmus, Tetraedriella, Tetraedron, Tetraselmis, Tetraspora, Tetrastrum, Thalassiosira, Thamniochaete, Thorakochloris, Thorea, Tolypella, Tolypothrix, Trachelomonas, Trachydiscus, Trebouxia, Trentepholia, Treubaria, Tribonema, Trichodesmium, Trichodiscus, Trochiscia, Tryblionella, Ulothrix, Uroglena, Uronema, Urosolenia, Urospora, Uva, Vacuolaria, Vaucheria, Volvox, Volvulina, Westella, Woloszynskia, Xanthidium, Xanthophyta, Xenococcus, Zygnema, Zygnemopsis, Zygonium, Chloroflexus, Chloronema, Oscillochloris, Heliothrix, Herpetosiphon, Roseiflexus, Thermomicrobium, Chlorobium, Clathrochloris, Prosthecochloris, Allochromatium, Chromatium, Halochromatium, Isochromatium, Marichromatium, Rhodovulum, Thermochromatium, Thiocapsa, Thiorhodococcus,Thiocystis, Phaeospirillum, Rhodobaca, Rhodobacter, Rhodomicrobium, Rhodopila, Rhodopseudomonas, Rhodothalassium, Rhodospirillum, Rodovibrio, Roseospira, Nitrobacteraceae sp., Nitrobacter sp., Nitrospina sp., Nitrococcus sp., Nitrospira sp., Nitrosomonas sp., Nitrosococcus sp., Nitrosospira sp., Nitrosolobus sp., Nitrosovibrio sp., Thiovulum sp., Thiobacillus sp., Thiomicrospira sp., Thiosphaera sp., Thermothrix sp., Hydrogenobacter sp., Siderococcus sp., Aquaspirillum sp. Methanobacterium sp., Methanobrevibacter sp., Methanothermus sp., Methanococcus sp., Methanomicrobium sp., Methanospirillum sp., Methanogenium sp., Methanosarcina sp., Methanolobus sp., Methanothrix sp., Methanococcoides sp., Methanoplanus sp., Thermoproteus sp., Pyrodictium sp., Sulfolobus sp., Acidianus sp., Bacillus subtilis, Saccharomyces cerevisiae, Streptomyces sp., Ralstonia sp., Rhodococcus sp., Corynebacteria sp., Brevibacteria sp., Mycobacteria sp., oleaginous yeast, Arabidopsis thaliana, Panicum virgatum, Miscanthus giganteus, Zea mays (plants), Botryococcus braunii, Chlamydomonas reinhardtii and Dunaliela salina (algae), Synechococcus sp PCC 7002, Synechococcus sp. PCC 7942, Synechocystis sp. PCC 6803, Thermosynechococcus elongatus BP-1 (cyanobacteria), Chlorobium tepidum (green sulfur bacteria), Chloroflexus auranticusl, Chromatium tepidum and Chromatium vinosum (purple sulfur bacteria), Rhodospirillum rubrum, Rhodobacter capsulatus, and Rhodopseudomonas palusris (purple non-sulfur bacteria).

It is claimed:

1. A metabolic process comprising:
a. introducing at least one substrate into a bioreactor containing an aqueous medium wherein the aqueous medium contains at least two biocatalysts wherein:
i. at least one of the biocatalysts is capable of bioconverting at least one substrate to at least one of an intermediate chemical and a sought chemical product,
ii. at least one other biocatalyst is capable of bioconverting at least one substrate or intermediate chemical to a chemical product or intermediate chemical product,
iii. at least one of said biocatalysts having
a solid structure of hydrated hydrophilic polymer defining an interior structure having a plurality of interconnected major cavities having a smallest dimension of between about 5 and 100 microns and an HEV of at least about 1000, and
a population of microorganisms substantially irreversibly retained in the interior structure, said microorganisms being in a concentration of at least about 60 grams per liter based upon the volume defined by the exterior of the solid structure when fully hydrated,
iv. at least one of the biocatalysts provides a chemical product;
b. maintaining the aqueous medium under metabolic conditions suitable for the bioconversion of said at least one substrate to at least one chemical product, and
c. recovering said at least one chemical product from the aqueous medium.

2. The process of claim 1 wherein at least two biocatalysts contain different microorganisms that are irreversibly retained therein.

3. The process of claim 2 wherein each of the biocatalysts contain microorganisms that are irreversibly retained therein.

4. The process of claim 2 wherein a biocatalyst contains at least two microorganisms.

5. The process of claim 4 wherein the biocatalyst is a layered biocatalyst.

6. The process of claim 1 wherein a first biocatalyst bioconverts at least one substrate to at least one intermediate chemical and at least one other biocatalyst bioconverts at least one intermediate chemical to at least one chemical product.

7. The process of claim 1 wherein a first biocatalyst bioconverts at least one substrate to at least one chemical product and at least one intermediate chemical and at least one other biocatalyst bioconverts at least one intermediate chemical to a further intermediate chemical or at least one chemical product.

8. The process of claim 7 wherein the further intermediate chemical is a substrate for another biocatalyst in the aqueous medium.

9. The process of claim 8 wherein the another biocatalyst is the first biocatalyst.

10. The process of claim 1 wherein at least two substrates are introduced into the aqueous medium, at least one of said substrates is bioconverted by at least one of said biocatalysts and at least one other of said substrates is not substantially bioconverted by said at least one biocatalyst but is bioconverted by at least one other biocatalyst.

11. The process of claim 10 wherein at least one substrate is bioconverted by at least one biocatalyst to at least one intermediate chemical and at least one intermediate chemical is bioconverted to chemical product by at least one other biocatalyst.

12. The process of claim 10 wherein the at least one substrate and the at least one other substrate are converted to the same chemical product.

13. The process of claim 10 wherein at least one substrate is bioconverted to at least one intermediate chemical by at least one biocatalyst, at least one other substrate is bioconverted to at least one other intermediate chemical by at least one other biocatalyst and the intermediate chemical and other intermediate chemical are bioconverted by at least one further biocatalyst to a chemical product.

14. A process for treating water containing disparate contaminants comprising:
(i) continuously introducing said water into a bioreaction zone containing a plurality of biocatalysts;
(ii) contacting the water with said biocatalysts under metabolic conditions for a time sufficient to reduce the concentration of the disparate contaminants; and
(iii) withdrawing water having a reduced concentration of disparate contaminants from the bioreaction zone containing a plurality of disparate contaminants,
wherein in said bioreaction zone a portion of the biocatalysts have substantially irreversibly retained therein one type of microorganism adapted to metabolically degrade at least one disparate contaminant, and at least one other portion of the biocatalysts have substantially irreversibly retained therein another type of microorganism adapted to metabolically degrade at least one other disparate contaminant, and wherein said biocatalysts comprise
a solid structure of hydrated hydrophilic polymer defining an interior structure having a plurality of interconnected major cavities having a smallest dimension of between about 5 and 100 microns and an HEV of at least about 1000, and
a population of microorganisms substantially irreversibly retained in the interior structure, said microorganisms being in a concentration of at least about 60 grams per liter based upon the volume defined by the exterior of the solid structure when fully hydrated.

15. The process of claim 14 wherein the disparate contaminants comprise at least one of metalates, nitrates and perchlorates that are subjected to reductive metabolic degradation and at least one of hydrocarbon and alkanol of from about 1 to 6 carbon atoms that are subjected to oxidative metabolic degradation, and other contaminants may be present.

16. The process of claim 14 wherein the water to be treated is produced water.

17. The process of claim 14 wherein the water to be treated comprises ground water that has been contaminated by subterranean fracturing for fossil fuel production.

18. The process of claim 14 wherein the bioreaction zone is a mobile bioreactor.

19. The process of claim 14 wherein the bioreaction zone is a point of use bioreactor.

20. A bioreactor for the bioconversion of at least one substrate to at least one chemical product comprising:
a. a vessel defining an interior volume;
b. an aqueous medium contained in at least a portion of the interior volume of the vessel; and
c. at least two biocatalysts distributed within the aqueous medium at least one of which comprises:
a solid structure of hydrated hydrophilic polymer defining an interior structure having a plurality of interconnected major cavities having a smallest dimension of between about 5 and 100 microns and an HEV of at least about 1000, and
a population of microorganisms substantially irreversibly retained in the interior structure, said microorganisms being in a concentration of at least about 60 grams per liter based upon the volume defined by the exterior of the solid structure when fully hydrated.
